(19) United States
(12) Patent Application Publication
(10) Pub. No.: US 2022/0081708 A1
(43) Pub. Date: Mar. 17, 2022

(54) DIAGNOSIS AND TREATMENT OF DYSBIOSIS-ASSOCIATED WITH NEC

(71) Applicant: EVOLVE BIOSYSTEMS, INC., Davis, CA (US)

(72) Inventors: Giorgio CASABURI, Sacramento, CA (US); Steven FRESE, Oakland, CA (US); Sufyan KAZI, Davis, CA (US)

(21) Appl. No.: 17/420,596

(22) PCT Filed: Jan. 4, 2020
(86) PCT No.: PCT/US2020/012277

§ 371 Date: Jul. 2, 2021

(51) Int. Cl. C12Q 1/689; G06N 20/00

(52) U.S. Cl. CPC C12Q 1/689; G06N 20/00

(57) ABSTRACT

This invention provides a method of determining risk of necrotizing enterocolitis (NEC) in an infant, comprising the steps of: (a) obtaining a fecal sample of the infant's relevant microbiome; (b) sequencing genetic material in the sample to obtain sequence data for the relevant microbiome; (c) analyzing sequence data for the relevant microbiome to identify biomarkers in the infant's microbiome; and (d) categorizing the NEC risk of the infant using the biomarkers identified in the microbiome of the infant.

### FIELD OF INVENTION

New machine learning tools or artificial intelligence (AI) are able to analyze key biomarkers including those from the fecal metagenome and metabolome to discriminate risk factors for disease in a variety of conditions and in particular preterm infants at risk of necrotizing enterocolitis (NEC).

### BACKGROUND

A major limitation in preventing or treating particular diseases is that a combination of genetics and environmental factors such as the composition and function of the host microbiomes including but not limited to the gut microbiome may be multifactorial and difficult to treat due to underlying variability in the functional capacity contained within the metagenome that may alter risk.

Prevention of a specific condition known to affect the preterm infant gut, neonatal necrotizing enterocolitis (NEC), dwells in the inability to predict which subset of premature infants is at risk for developing NEC. Recently, gut dysbiosis has emerged as a major trigger in NEC, particularly supported by the fact that NEC cannot be produced in germ free animals.

Major limitations have been encountered when focusing solely at the taxonomic level. Composition of the microbiome (i.e., which microbial species are represented) is not enough to be able to uncover microbial signatures for NEC. A greater depth of functional information is required to be able to uncover the patterns required for accurately diagnosing and altering the microbiome function to correct for the risk a premature infant has of developing NEC.

### SUMMARY OF INVENTION

This invention provides a method of determining risk of necrotizing enterocolitis (NEC) in an infant, comprising the steps of: (a) obtaining a fecal sample of the infant's relevant microbiome; (b) sequencing genetic material in the sample to obtain sequence data for the relevant microbiome; (c) analyzing sequence data for the relevant microbiome to identify biomarkers in the infant's microbiome; and (d) categorizing the NEC risk of the infant using the biomarkers identified in the microbiome of the infant.

In a preferred mode, the categorizing according to step (d) is based on an artificial intelligence (AI) model developed by analyzing sequence data from the relevant microbiomes of N infants, the N infants comprising at least M infants diagnosed with NEC, and N−M infants not diagnosed with NEC, where the AI model is developed by processing the sequence data from the relevant microbiomes of the N infants by Machine Learning algorithms to identify at least X biomarkers which differ significantly between infants diagnosed with NEC and infants not diagnosed with NEC and associating the X biomarkers with infants having or at risk for having NEC. Generally, N is at least 10-fold higher than X and M is at least 2-fold higher than X. Preferably, N is between 400 and 10,000 infants, and M is between 200 and 1300 infants, and more preferably, X is at least 5, at least 10, at least 20, at least 30 or at least 40 biomarkers. Typically, the biomarkers identified in step (c) are proteins, mobile genetic elements, functional annotations, superpathways, taxonomic identifiers, and/or combinations thereof. Preferably, the biomarkers identified in step (c) are biomarkers found on Table 5 and/or 6.

In accordance with this invention, the infant may be a term infant or a preterm infant. The relevant microbiome for this invention may be an intestinal microbiome, fecal microbiome, a milk microbiome, a skin microbiome, an environmental microbiome, or a combination thereof. Further according to this invention, the infant's risk of NEC is likely to be categorized as high if intestinal ARG levels are low [add quantitiation], and/or the [insert quantifiable threshold for intestinal integrity]. This invention also provides for therapy of an infant having high risk of NEC categorized according to this invention, where such infants are treated by administering B. infantis and/or mammalian milk oligosaccharides (MMO).

### DESCRIPTION OF FIGURES

FIG. 1. Ideal corrected gestational age (cGA) window discriminates NEC microbiome signatures from preterm controls (no NEC)

FIG. 2. Comparison of the sensitivity and specificity across different machine learning models derived from superpathways classification to select for the best model.

FIG. 3. Most discriminative bacterial species identified in the AI model

FIG. 4. Mean relative abundance of Bifidobacteriaceae with the 29-32 cGA window is generally lower in NEC samples compared to control (no NEC) samples

FIG. 5 Mean relative abundance of Bifidobacterium longum with the 29-32 cGA window is generally lower in NEC samples compared to control (no NEC) samples

FIG. 6 Mean relative abundance of Enterobacteriaceae with the 29-32 cGA window is generally higher in NEC samples compared to control (no NEC) samples

FIG. 7. Mean relative abundance of Enterobacter cloacae with the 29-32 cGA window is generally higher in NEC samples compared to control (no NEC) samples

FIG. 8. Microbiome-mediated arginine (Arg) metabolism pathways differ in NEC cases compared to preterm controls (no NEC). EC numbers are used to represent enzymes. *** highest fold change in NEC compared to control, ** next highest group. * 3^{rd} highest group, # decreased in NEC compared to control.

FIG. 9. Different bacterial species contribute to arginine depletion in NEC cases vs preterm controls (no NEC)

### DETAILED DESCRIPTION OF THE INVENTION

Inventors have developed a process for characterizing microbiome samples which reveals a biomarker pattern associated with NEC. This process can be utilized with any human-associated microbiome, including but not limited to, fecal, skin, or milk, as well as environmental microbiome such as those found on non-living surfaces or in the air, to assess the likelihood of the presence of NEC in the individual or the likelihood of development of NEC. This process could further be utilized to assess the risk of development of NEC by patients exposed to environments shown to exhibit a NEC-associated biomarker pattern.

This process consists primarily of the collection of a microbiome sample, followed by analysis of said sample through genetic sequencing techniques; resulting sequence data is then annotated by labeling genes associated with microbial biomarkers and superpathways. Annotated sequence data is further analyzed through one or more machine learning algorithms which have been trained to detect biomarker and superpathway patterns associated with NEC.

Indifferent to host genetic background, AI or machine learning offers the potential to provide previously undiscovered associations that facilitate stratification of risk within a particular population to identify not only individuals most at risk, but also to provide alternative protocols and therapies that can be deployed to prevent and/or treat based on these different risk profiles.

The insights from machine learning can be used to provide a deeper, more complete understanding of interactions and critical influencers within the microbiome that are a signature of the underlying dysbiosis associated with NEC. Applications can include a new drug discovery pipelines, environmental monitoring, new treatment protocols for prevention and/or treatment options that focus on risk reduction.

Fecal samples provide an underexplored opportunity to non-invasively understand a number of systems simultaneously, including metabolic, immune activity, and intestinal integrity. Intestinal integrity includes proliferation or growth, wound healing, tight junctions, mucin production, and/or immune activity as a measure of competence against dysbiosis-associated disease conditions.

The invention described here goes beyond taxonomic classification to be agnostic on the precise composition of the gut microbiome but rather focuses on the functional capacity down to the individual gene level to predict with better accuracy the NEC risk and treatment options. The specific biomarker patterns and/or superpathways provide a more integrated, comprehensive, and holistic view of the gut microbiome and its function that can be monitored.

The algorithm can be used on unknown samples from infants in the NICU by taking a fecal sample and sequencing the fecal sample using shotgun metagenomics, which will allow taxonomic and functional characterization of the infant's microbiome. The sequencing data is then entered into the software assembled as part of this invention in which an algorithm is used to predict NEC risk.

Moreover, coupling metagenomics with metabolomics, observed as well as predicted via machine learning, will identify proteins that are signatures of NEC risk. This platform may be used to identify the biomarkers and then develop assays based on the knowledge of the bacteria present, the gene functions, gene expression, protein expression, and/or the output of one or more key metabolites in identified superpathways

The protein biomarkers may be used to create a protein-based assay, which may be employed to indicate the level of NEC risk before proceeding with shotgun metagenomic sequencing and may also lead to small molecule drug discovery through a greater understanding of the metabolomics profile. The protein assay may provide a rapid diagnostic tool aiding doctors in deciding how to handle each case of prematurity and greatly reduce errors in communication or individual diagnosis.

These may also be used to develop new drug candidates to sort through the abundance of the gene products most often associated with NEC.

Necrotizing enterocolitis (NEC) mostly affects the intestine of premature infants, but may affect term infants with other conditions. The wall of the intestine is invaded by bacteria, which cause local infection and inflammation that can ultimately destroy the wall of the intestine. Portions of the intestine die. The disease has three stages:
Bell's stage 1 (suspected disease):
   Mild systemic disease (apnea, lethargy, slowed heart rate, temperature instability);
   Mild intestinal signs (abdominal distention, increased gastric residuals, bloody stools);
   Non-specific or normal radiological signs.
Bell's stage 2 (definite disease):
   Mild to moderate systemic signs;
   Additional intestinal signs (absent bowel sounds, abdominal tenderness);
   Specific radiologic signs (pneumatosis intestinalis or portal venous gas;
   Laboratory changes (metabolic acidosis, too few platelets in the bloodstream).
Bell's stage 3 (advanced disease):
   Severe systemic illness (low blood pressure);
   Additional intestinal signs (striking abdominal distention, peritonitis);
   Severe radiologic signs (pneumoperitoneum);
   Additional laboratory changes (metabolic and respiratory acidosis, disseminated intravascular coagulation).

NEC burst. A period where the incidence of NEC spikes in the NICU seasonally due to an unknown change in the environment, probably linked to change in the microbial community composition.

Preterm infant is defined as babies born alive before 37 weeks of pregnancy are completed. There are sub-categories of preterm birth, based on gestational age: extremely preterm (less than 28 weeks) very preterm (28 to 32 weeks) moderate to late preterm (32 to 37 weeks). These infants may also be classified according to birth weight. Infants born with a birth weight less than 1500 g are defined as very low birth weight (VLBW) infants. Low birth weight (LBW) is defined as a birth weight of less than 2500 g (up to and including 2499 g).

Metagenome or metagenomic profile is defined as the totality of the DNA recovered from a given biological sample that can include human, bacteria, viruses, mold and yeast DNA.

Skin microbiome is any microbiome that can be recovered from any skin surface.

Milk microbiome is collected by swabbing the breast and is considered the extension of the maternal skin and infant buccal microbiomes.

Environmental microbiome refers to a sample containing the collection of microorganisms retrieved from any environmental source, including but not limited to, non-living surfaces; air; food; and/or water.

Dysbiosis-associated disease condition (DADC). A DADC refers to any physiological condition associated with an unhealthy composition and/or function of the individual's gut microbiome.

Metabolomic profile is the sum of all metabolites measured at a given time to provide a snapshot of overall metabolic output. It may be relative between one group or the next or may be quantified.

Superpathways are groups of functionally related reactions and/or metabolic or biosynthetic pathways.

Biomarker is any genetic information or information obtained by analyzing a genome. They include proteins, mobile genetic elements, functional annotations, superpathways, and taxonomic information among others.

Oligosaccharide refers to polymeric carbohydrates that contain 3 to 20 monosaccharides covalently linked through glycosidic bonds. In some embodiments, the oligosaccharides are purified from human or bovine milk/whey/cheese/dairy products, {e.g., purified away from oligosaccharide-degrading enzymes in bovine milk/whey/cheese/dairy products).

Mammalian milk oligosaccharides are oligosaccharide compounds found, but not necessarily exclusively found, in mammalian milk. Mammalian milk oligosaccharides may come from any source so long as they are analogous in structure and/or function to those found in mammalian milk.

Synthetic human milk products containing prebiotics are those that are processed for delivery to the premature infant. Processing may occur in a manner which serves to preserve the milk and/or alter the composition. Pasteurization, or other heating methods) freezing, fractionation, separation and reassembly may all be considered. A prebiotic product may be any product that has at least one mammalian milk oligosaccharide of any species (i.e., human, bovine, ovine) contained in infant formula, or as a standalone product that is then mixed with human milk or infant formula, water or other liquid suitable for the preterm infant. The mammalian milk oligosaccharide may be derived from a synthetic process in yeast, or E. coli or other chemical synthesis as long as it has a structure that matches the structure or function of human milk. Examples include, but are not limited to Lacto-N-biose, Lacto-neotetraose (LNT), Lacto-N-neotetraose (LNnT), Fucosyl lactose (2″FL or 3′FL), Sialyl lactose (3′SL or 6″SL).

As described below, the input for the analysis may be metagenome DNA sequences pulled from other databases and properly curated before analysis.

Typically, the input starts with collection of microbiome samples which may be fecal samples. Fecal samples are non-invasive and can be readily collected from vulnerable populations, including but not limited to preterm infants and other hospitalized groups. DNA sequencing of fecal samples for preterm patient populations who may or may not be at risk for NEC can be used to better stratify the population by identifying those individuals who are at risk for development of a DADC (such as NEC) to improve the effectiveness of protocols or therapies used to treat patients under physician care. This can be achieved by isolating the total DNA present in fecal samples that includes all the human, bacteria, viruses, yeast and mold present in that sample. The DNA can be prepared for deep sequencing that allows for all of the different contributions to be detected. The inventors also utilized a tool (bowtie2) to scrub all human DNA from the analysis for HIPPA compliance which renders de-identified samples for further population-based analysis, when required.

Metagenomics analysis of microbiome samples (e.g., fecal samples) can be used to understand key differences between certain groups. Certain embodiments of the invention provide a method of measuring the metagenome to identify differences between individuals in a given group. The group may consist of individuals within the same age group with unknown or known risk factors for a certain condition. In some embodiments, the metagenome is used in the method to help identify differences between individuals or to determine health status of an individual. It is also possible to take repeated measures from the same individual over time to assess pre-clinical differences between individuals who later went on to develop the condition. This metagenomic approach can be used to both better describe the condition, but also to look for earlier warning signs to be able to provide more effective treatment.

In some embodiments, the metagenome information is combined with other microbial data such as the fecal metabolomic data, which may be a combination of microbial and host metabolites. Other host information from fecal samples, such as cytokine data, may be added to the machine learning model to see additional interactions and determine what are the most significant influencers concerning either the presence or absence of NEC. Further, the host information may be used to determine if these most significant influencers change whether the sample is from an infant with stage 1, 2 or 3 NEC.

It is recognized that in some embodiments only a subset of the detected differences are clinically significant and that the data may be prioritized and or limited based on a number of different markers; these markers may be part of key superpathways, and the superpathways may be defined as key metabolites, key enzyme activities and/or presence of key proteins to assess risk or by certain gene products.

It is also recognized that in some embodiments, the time frame for metagenomics may not be practical for the treatment of individuals but may be an effective strategy to evaluate specific population risk and also to evaluate the success of any risk mitigation strategy deployed in a healthcare setting. However, taking a subset of metabolites, bacteria, or proteins identified as part of the metagenomic analysis that are key risk factors can be developed into lab tests or more preferably point of care tests that provide information to evaluate the risk of a particular disease in a particular individual receiving treatment. The application of these tests provides a strategy for personalizing treatment protocols and therapies to suit individual needs.

It is also recognized that a subset of the metagenome and metabolomic analysis may be used to assess specific gut functions including but not limited to intestinal integrity. Intestinal integrity is a general term that may include factors such as tight junction integrity, wound healing capacity, mucus layer integrity, and/or bacterial translocation.

It may also be used to establish appropriate gut motility that may be measured as stooling patterns, number of stools per day and/or stool consistency.

In yet other embodiments, particular subsets maybe used to control treatment of certain conditions or used to prevent certain conditions or symptoms in individuals. In some embodiments, the treatment of the individual first requires diagnostic and/or prognostic characterization.

### Development of the AI Model

A non-invasive approach that combined functional and taxonomical data from infant fecal samples was used to evaluate infant gut microbiomes and to develop an artificial intelligence (AI) model able to predict significant metagenomic biomarkers of NEC among a preterm infant population.

Cohort selection and data extraction. A total of eight studies were selected that performed shotgun metagenomic sequencing matching the word “NEC” or “preterm” on NCBI Sequence Read Archive (SRA). A summary of the studies and patient characteristics can be found in Table 1. In order for a sample to be included in the analysis a minimum of intrinsic metadata criteria had to be met in regard to reporting “day of life”, “NEC presence/absence”, “antibiotic treatment”, “country of origin”, “gestational age”, “delivery mode”, “feeding practice”, “sex” and “birth weight”. After applying filtering criteria based on meta data, a total of 1,647 shotgun metagenomic raw datasets were retained. These represent every shotgun metagenomics sequencing dataset from preterm babies available in the NCBI SRA.

TABLE 1
Summary of sources of metagenomic information and patient characteristics

| | Gestational | | | | | |
|---|---|---|---|---|---|---|
| # of | age at birth | | | | | |
| Samples | (Week) | Sex | Country | NEC | Diet | Study |
| 15 | 24.4 | n/a | UK | NO | n/a | Rose G, 2017 |
| 141 | 27.3 | 37% F | USA | NO | mix | Raveh-Sadka T, 2016 |
| 369 | 27 | 59% F | USA | NO | mix | Gibson MK, 2016 |
| 37 | 26.3 | n/a | USA | NO | mix | Olm MR, 2017 |
| 398 | 26.4 | 39% F | USA | 18% | mix | Brooks B, 2017 |
| 283 | 29.1 | 60% F | USA | 17% | mix | Rahman, 2018 |
| 357 | 26.3 | 7% F/81% | USA | 17% | n/a | Taft DH, 2014 |
| | | n/a | | | | |
| 47 | 29.2 | 21% | USA | 62% | mix | Raveh-Sadka T, 2015 |

Feature annotation. Samples were analyzed concurrently within the same pipeline. Taxonomic profiling of the metagenomic samples was performed using MetaPhlAn2[Truong D T, Franzosa E A, Tickle T L, Scholz M, Weingart G, Pasolli E, Tett A, Huttenhower C, Segata N. 2015. MetaPhlAn2 for enhanced metagenomic taxonomic profiling. Nature methods 12:902] with default parameters, using the included library of clade-specific markers to provide panmicrobial (bacterial, archaeal, viral and eukaryotic) profiling. Functional gene characterization was performed using the Humann2 [Franzosa E A, McIver L J, Rahnavard G, Thompson L R, Schirmer M, Weingart G, Lipson K S, Knight R, Caporaso J G, Segata N. 2018. Species-level functional profiling of metagenomes and metatranscriptomes. Nature methods 15:962.] pipeline with default settings following the updated global profiling of the Human Microbiome Project analysis pipeline (2017) [Lloyd-Price J, Mahurkar A, Rahnavard G, Crabtree J, Orvis J, Hall A B, Brady A, Creasy H H, McCracken C, Giglio M G. 2017. Strains, functions and dynamics in the expanded Human Microbiome Project. Nature. After running samples through MetaPhlan and Humann2 pipeline, matrices were obtained containing taxonomic or functional annotations based on different classifications against Uniref90 [Apweiler R, Bairoch A, Wu C H, Barker W C, Boeckmann B, Ferro S, Gasteiger E, Huang H, Lopez R, Magrane M. 2004. UniProt: the universal protein knowledgebase. Nucleic acids research 32:D115-D119], KEGG [Kanehisa M, Goto S. 2000. KEGG: kyoto encyclopedia of genes and genomes. Nucleic acids research 28:27-30] and MetaCyc. [Caspi R, Foerster H, Fulcher C A, Kaipa P, Krummenacker M, Latendresse M, Paley S, Rhee S Y, Shearer A G, Tissier C. 2007] databases. The MetaCyc Database of metabolic pathways and enzymes and the BioCyc collection of Pathway/Genome Databases. Nucleic acids research 36:D623-D631].

Statistical analysis. Significantly different genes among treatments were estimated using the Kruskal-Wallis one-way analysis of variance, coupled with FDR or Bonferroni correction as cross-sample normalization. A Bray-Curtis dissimilarity matrix was constructed to estimate global differences among samples and visualized via Principal Coordinate Analysis (PCoA). Permutational Multivariate Analysis of Variance Using Distance Matrices (adonis) was used to assess global microbiome differences between groups. P-value for PCoA panel was computed using F-tests based on sequential sums of squares from permutations of the raw data. P-values throughout this analysis are represented by asterisks (*, P<0.05; **, P<0.01; ***, P<0.001; ****, P<0.0001).

A total of 1,712 raw publicly available shotgun metagenomic datasets were collected (NEC=253; and healthy preterm=1,459) and entered into a data analysis pipeline that consists of a number of processing steps that can be analyzed concurrently within the same pipeline that results in meaningful outputs on the metagenomic data set. Taxonomic profiling of the metagenomic samples was performed using MetaPhlAn2 with default parameters, using the included library of clade-specific markers to provide panmicrobial (bacterial, archaeal, viral and eukaryotic) profiling. Functional gene characterization was performed using the Humann2 pipeline with default settings following the updated global profiling of the Human Microbiome Project analysis pipeline. After MetaPhlan and Humann2 pipelines, a plurality of different matrices were obtained containing taxonomic or functional annotations based on different classifications against Uniref90, KEGG, and MetaCyc databases. After quality filtering of sequence datasets, a subset of the data (n=1,647) was selected for downstream analysis. The dataset was divided based on corrected gestational age (cGA) according to NEC occurrence. This dataset was the input for several artificial intelligence (AI)/machine learning models (Random Forest and Gradient Boosting classifiers). The different models were used to identify functional core biomarkers able to distinguish NEC from healthy preterm infant microbiomes.

Data preparation and feature engineering. An initial two datasets, an unstratified pathway abundance dataset and a pathway abundance dataset stratified by bacterial species, were divided into smaller datasets by corrected gestational age (cGCA). Each dataset was divided into samples with cGCA lower than 29 weeks and samples with cGCA 29 weeks or higher. Each of these four datasets was further divided into four smaller datasets: a training set with original NEC distribution, a training set with oversampled NEC distribution, a testing set (20%) of unique samples, and a validation set (20%) of unique samples.

Machine Learning. A decision tree is a common classification model where, to classify the target, the optimal split from the optimal feature is serially made to maximize accuracy (or some other metric). This results in a hierarchical model where each node is used as a filter until a sample is classified. Random forests are ensembles of individual decision trees where voting is implemented to determine the final prediction of the ensemble and only a subset of random features is considered for each optimal split in each tree. Thus, each composing tree is significantly different from all others in the model and captures a different signal from the data upon which it is trained.

A Gradient Boosting Classifier is similar to a random forest, however it determines the criterion for splitting by a feature by creating and minimizing a differentiable loss function of the entire tree. It then tunes these values with subsequently smaller tweaks and aggregating all trees into an ensemble.

For each training dataset, a Random Forest Classifier and a Gradient Boosting Classifier were trained from python's scikit-learn library. Models were trained to predict NEC occurrence from stratified and unstratified bacterial superpathways from each of the 8 datasets. Hyperparameters for a gradient boosting classifier and random forest classifier were grid-searched for each dataset resulting in the final 16 models.

The Ideal Hyperparameters for the Random Forest Model Through Grid-Search

For each Random Forest model, the following hyperparameters were tested. Bootstrap was set to ‘True’. Max depth was grid-searched for each dataset between 1, 2, 3, 5, 8, 12, and ‘None’. The number of estimators was set to 500. Random state was set to 310 and all other hyperparameters were left at scikit-learn's default values. For each Gradient Boosting model, the learning rate was grid-searched for each dataset across 0.1, 0.15, 0.2, and 0.3, the max depth across 1, 2, 3, 4, 5, 6, and ‘None’, and the minimum number of samples per leaf across 1, 2, 3, and 4. The number of estimators was also set to 500. Random state was set to 310 and all other hyperparameters were left at scikit-learn's default values. Feature importances were calculated from the highest performing hyperparameters using Gini importance scores. Because Gini importance scores account for the impurity at each node, these scores were expected to change significantly between the balanced and unbalanced datasets. Thus, to confirm findings from feature importance scores permutation importances were also calculated on the test dataset and compared.

Ranking. A sublist of statistically significant proteins was obtained by conducting a Kruskal-Wallis test with each protein. Protein feature ranking of Uniref_90 proteins was determined by conducting recursive feature elimination on a random forest classifier. Approximately 6.1 million proteins were filtered by conducting a Kruskal-Wallis test with each protein, including only the 3420 statistically significant features. A feature ranking of these Uniref_90 proteins was determined by conducting recursive feature elimination on a random forest classifier.

Scikit-learn's Recursive Feature Elimination algorithm was implemented where the hyperparameters for the most performant model identified through grid-search were utilized. A train, test, and validation accuracy score was calculated for each set of top ranked features. Thus, the minimum number of features required to obtain consistent maximal accuracy was determined. A model was then trained utilizing the ideal hyperparameters previously identified and was tested on two holdout datasets.

As a comparison, a random forest model was trained on the full feature-set of the gene families dataset with a train:test:validation split of 60:20:20. A machine with 468 GB of RAM and 64 cores was utilized. The hyperparameters utilized were n_estimators=300, max_depth=None, random_state=310 and oob_score=True.

### Results

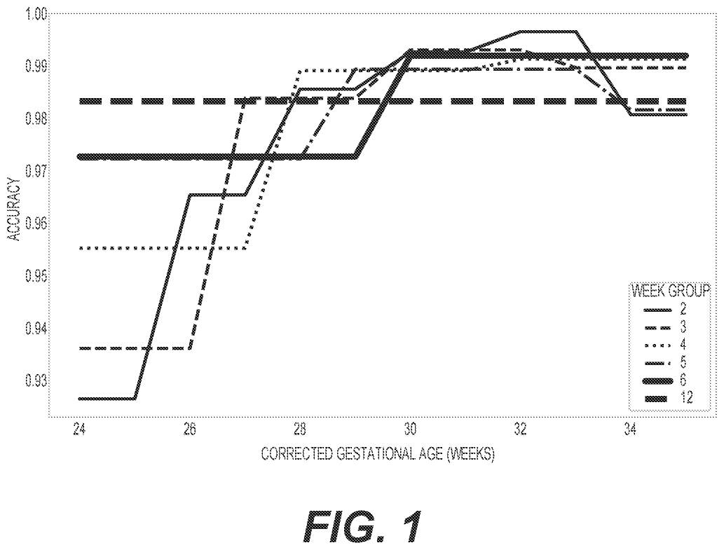

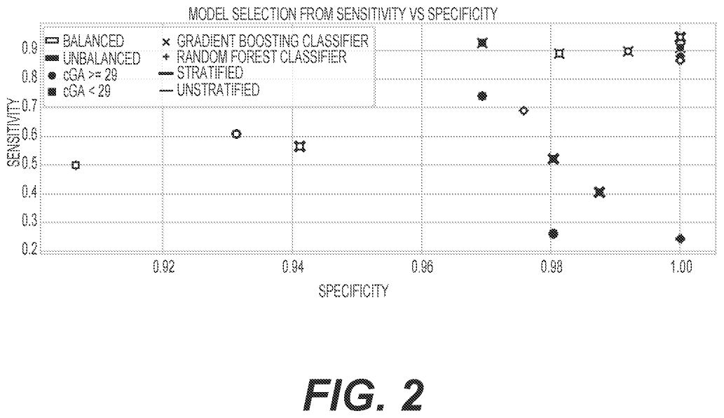

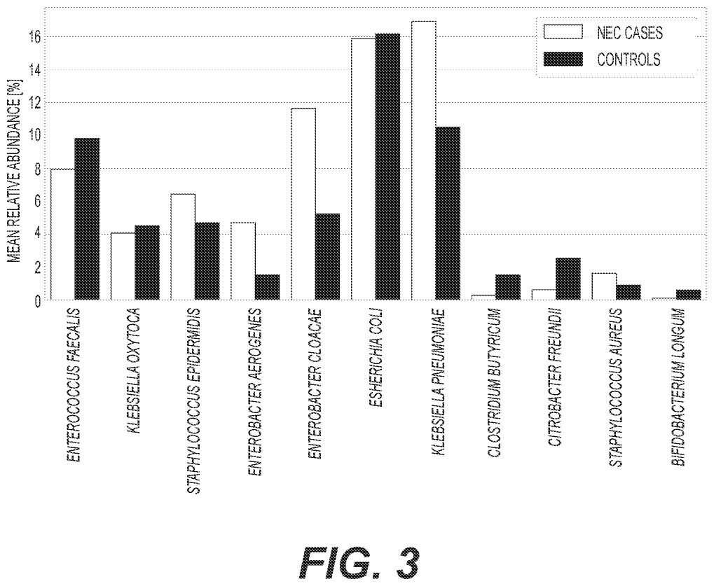

Globally, 928 different microbial species were identified (4 Archeae; 9 Eukaryota; 7 Viroids; 397 Bacteria; 511 Viruses). FIG. 1 identified a critical window for NEC. The 29-32 weeks cGA population reported a significant level of prediction accuracy among models (up to 99.8%). Intersection of the different models led to the identification of top proteins and superpathways, which were then coupled with taxonomic classification to establish a collection of biomarkers, in particular the bacterial species, able to discriminate NEC from healthy preterm infants. The most performant models were identified by plotting the sensitivity and specificity of the testing datasets (FIG. 2). Models built from stratified pathways and samples with a corrected gestational age greater than or equal to 29 weeks consistently performed higher than others. Additionally, gradient boosting classifiers performed nominally better in sensitivity when compared with random forest models. The most discriminatory microbial species among samples were identified (see FIG. 3).

Besides taxonomic profiling we were able to characterize the functional microbiome in terms of protein coding genes as well as superpathways. Gene family entries were converted into pathways. By default, HUMAnN2 uses MetaCyc pathway definitions and MinPath to identify a parsimonious set of pathways that explain observed reactions in the community. This led to a matrix of 1,605 (samples)×19,039 (pathway) or 30.5 million entries. First, Principal Component Analysis (PCA) was used to investigate our data set both across taxonomic and gene features. This revealed insights into the structure of the data from both a sample and a feature perspective.

Second, we divided the sampling size into different subsets based on corrected gestational age and applied random forest techniques to assess whether the NEC or healthy preterm status could be predicted based on microbiome signatures. Since there is no previous indication on which microbial feature should be over or under abundant in NEC vs. healthy preterm state, we used the Kruskal-Wallis test to determine the subset of gene families that are most statistically significant between NEC and healthy preterms. From the Kruskal-Wallis test we selected entries with an adjusted p<0.0001 (Bonferroni). The 3,420 significant gene families were then converted into KEGG functional orthologs (KO), resulting in 155 KO features (Table 3). The 3,420 gene families were further analyzed to look for redundant functions. For instance, if the same enzyme was identified from two different bacteria, this would give two different gene family entries from the UniProt database but converted in KEGG would result in one KO entry (namely an ortholog with same function independently from its taxonomic origin). Any KO might consist of multiple UniProt with the commonality of being related by vertical descent from a common ancestor and encoding proteins with the same function in different species. Therefore, we have determined the most statistically significant over and under abundant KEGGs in NEC state.

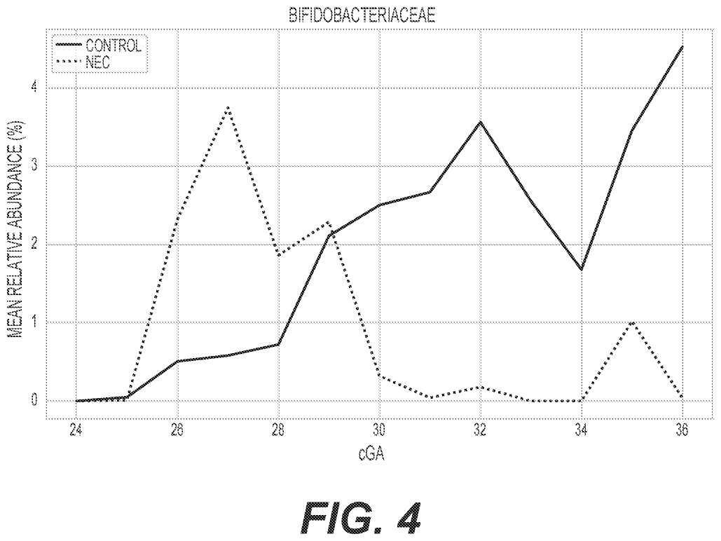

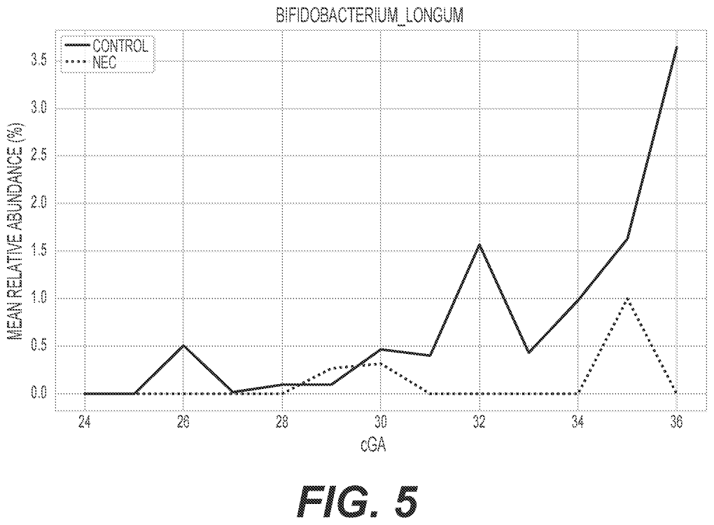

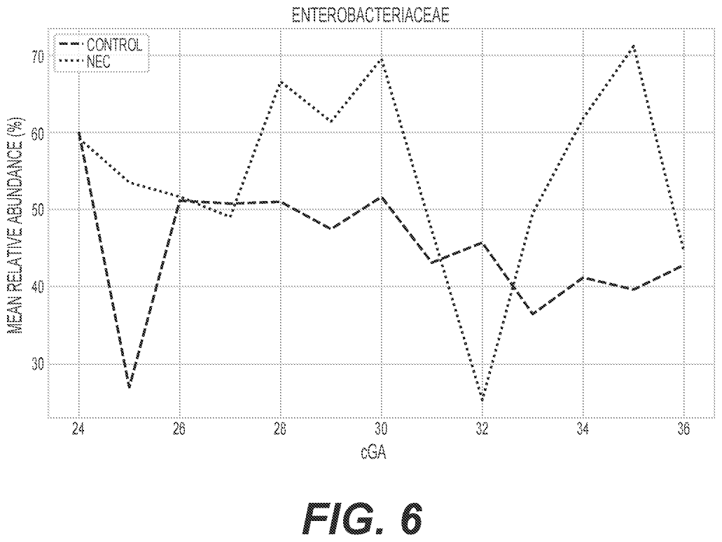

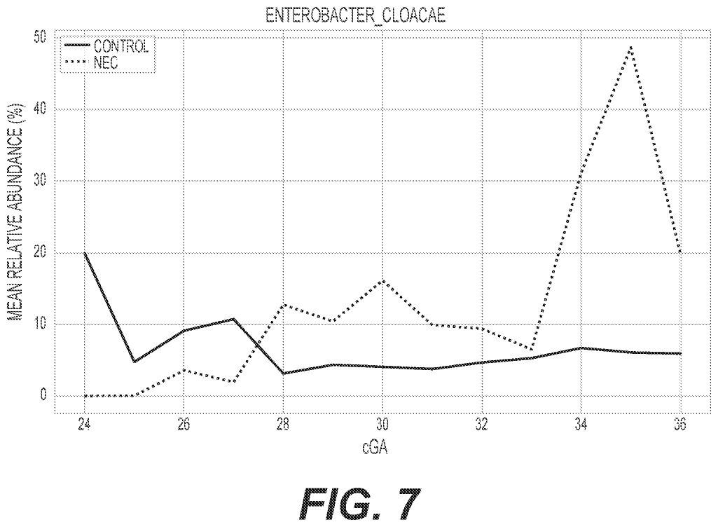

Bifidobacteriaceae were lower in infants with NEC and this was also true for Bifodobacterium longum (B. longum) that includes the subspecies B. longum subsp. infantis (B. infantis). In contract Enterobacteriaceae and in particular, Enterobacter clocae (FIGS. 4-7, respectively)

The data set was further evaluated and here we report an example of some significant proteins (Table 2), KEGG gene orthologs (Table 3) identified among samples.

TABLE 2
Most significant proteins identified for 29-32 cCGA composition
identified via Humann2. Statistical significance is expressed in
P-values computed via Kruskal-Wallis ANOVA.

| UniProt Protein ID | P-value | NEC_mean | Preterm_mean |
|---|---|---|---|
| UniRef90_J7GDE2 | 4.12E−32 | 6.13E−06 | 4.11E−08 |
| UniRef90_A5IR78 | 3.25E−28 | 6.88E−06 | 2.16E−09 |
| UniRef90_Q8SDU6 | 5.38E−28 | 1.29E−06 | 1.41E−08 |
| UniRef90_G8C7S1 | 5.23E−27 | 3.53E−06 | 8.55E−08 |
| UniRef90_A6QI72 | 5.51E−27 | 6.43E−06 | 4.67E−09 |
| UniRef90_J7G874 | 1.26E−26 | 4.93E−06 | 4.92E−09 |
| UniRef90_B5XNT5 | 2.37E−26 | 1.68E−06 | 2.06E−07 |
| UniRef90_Q8SDT6 | 5.92E−26 | 5.57E−06 | 4.35E−09 |
| UniRef90_Q8SDM3 | 6.49E−26 | 6.48E−06 | 1.29E−09 |
| UniRef90_Q8SDU9 | 7.27E−26 | 6.30E−06 | 7.24E−09 |
| UniRef90_Q8SDV0 | 8.96E−26 | 6.76E−06 | 4.37E−09 |
| UniRef90_Z2VPU9 | 9.48E−26 | 1.08E−07 | 1.55E−10 |
| UniRef90_B2ZYY5 | 1.21E−25 | 1.01E−06 | 1.49E−09 |
| UniRef90_N5LAZ0 | 1.21E−25 | 4.70E−07 | 8.87E−10 |
| UniRef90_A6QI70 | 1.25E−25 | 6.34E−07 | 1.51E−09 |
| UniRef90_J7GF25 | 3.53E−25 | 4.43E−06 | 1.33E−08 |
| UniRef90_B2ZYZ1 | 8.15E−25 | 6.39E−06 | 1.38E−08 |
| UniRef90_J7G9K4 | 8.35E−25 | 4.60E−06 | 2.77E−09 |
| UniRef90_M9NSW2 | 9.34E−25 | 6.45E−06 | 3.43E−09 |
| UniRef90_A0A019VBT6 | 1.67E−24 | 3.67E−07 | 5.22E−10 |
| UniRef90_N5CYX6 | 1.85E−24 | 9.14E−07 | 4.85E−10 |
| UniRef90_A6QG13 | 1.92E−24 | 6.55E−06 | 3.61E−09 |
| UniRef90_A0A008NE55 | 2.16E−24 | 1.78E−06 | 8.34E−10 |
| UniRef90_J7GE72 | 2.78E−24 | 4.31E−06 | 8.11E−09 |
| UniRef90_J7GN81 | 8.99E−24 | 2.36E−06 | 6.17E−09 |
| UniRef90_J7GDT7 | 1.57E−23 | 5.70E−06 | 2.35E−09 |
| UniRef90_C3R384 | 1.73E−23 | 1.33E−05 | 0 |
| UniRef90_D4UIW5 | 1.73E−23 | 3.83E−06 | 0 |
| UniRef90_N1N3C6 | 1.73E−23 | 1.83E−06 | 0 |
| UniRef90_Y8A8R7 | 2.21E−23 | 4.36E−07 | 1.84E−09 |
| UniRef90_Y1EIY8 | 2.30E−23 | 5.97E−06 | 2.82E−09 |
| UniRef90_W5VJZ3 | 2.92E−23 | 1.22E−06 | 7.92E−10 |
| UniRef90_S3ACE4 | 2.94E−23 | 9.58E−06 | 1.28E−07 |
| UniRef90_Y9N0L4 | 3.25E−23 | 1.22E−06 | 2.77E−10 |
| UniRef90_D6DXM7 | 3.43E−23 | 1.37E−05 | 1.52E−06 |
| UniRef90_V0XLH8 | 4.29E−23 | 4.74E−07 | 5.21E−09 |
| UniRef90_A5IR66 | 1.11E−22 | 6.29E−06 | 4.62E−09 |
| UniRef90_A6QDW5 | 1.47E−22 | 6.18E−06 | 4.20E−08 |
| UniRef90_J7GEH7 | 1.58E−22 | 4.54E−06 | 3.03E−09 |
| UniRef90_A6QI74 | 1.93E−22 | 6.86E−06 | 1.55E−08 |
| UniRef90_A5IR71 | 2.54E−22 | 7.04E−06 | 2.36E−08 |
| UniRef90_A5IR73 | 2.58E−22 | 6.78E−06 | 5.00E−09 |
| UniRef90_A6QG07 | 2.76E−22 | 6.63E−06 | 4.27E−09 |
| UniRef90_V3DLZ1 | 2.82E−22 | 1.54E−06 | 1.78E−08 |
| UniRef90_Y1HC02 | 3.00E−22 | 1.05E−06 | 8.87E−09 |
| UniRef90_A6QI68 | 3.10E−22 | 5.62E−06 | 9.13E−09 |
| UniRef90_S3AS97 | 3.10E−22 | 6.10E−06 | 0 |
| UniRef90_B5XZ53 | 3.45E−22 | 2.57E−06 | 5.84E−08 |
| UniRef90_J7GEU9 | 4.83E−22 | 4.74E−06 | 4.00E−09 |
| UniRef90_Q4ZDW4 | 4.94E−22 | 6.83E−06 | 5.36E−10 |
| UniRef90_J7GIK8 | 5.40E−22 | 4.05E−06 | 6.40E−09 |
| UniRef90_B7T0C8 | 5.99E−22 | 2.22E−06 | 1.43E−09 |
| UniRef90_G8V2M3 | 6.81E−22 | 2.01E−05 | 2.96E−08 |
| UniRef90_G2SBG8 | 7.22E−22 | 1.27E−05 | 1.46E−07 |
| UniRef90_Z0ATC5 | 7.48E−22 | 2.54E−07 | 1.55E−09 |
| UniRef90_UPI00036C4590 | 7.62E−22 | 4.22E−05 | 7.57E−09 |
| UniRef90_N5HUQ2 | 7.73E−22 | 3.75E−07 | 2.03E−09 |
| UniRef90_Q7X238 | 1.04E−21 | 5.07E−06 | 4.17E−09 |
| UniRef90_V3D1P5 | 1.49E−21 | 1.11E−07 | 1.87E−09 |
| UniRef90_J7GAZ0 | 1.50E−21 | 4.11E−06 | 1.50E−08 |
| UniRef90_X1WTI2 | 1.53E−21 | 4.53E−06 | 5.48E−08 |
| UniRef90_Q8SDU3 | 1.71E−21 | 7.00E−06 | 5.19E−09 |
| UniRef90_D2ZH17 | 3.25E−21 | 7.47E−07 | 5.59E−09 |
| UniRef90_YOGIW0 | 3.43E−21 | 1.02E−06 | 3.72E−09 |
| UniRef90_G2S602 | 3.66E−21 | 1.05E−05 | 3.07E−07 |
| UniRef90_I0TMD8 | 3.67E−21 | 7.98E−06 | 2.53E−07 |
| UniRef90_J7GJ86 | 3.77E−21 | 5.26E−06 | 1.96E−08 |
| UniRef90_S2ZTB6 | 4.11E−21 | 1.07E−05 | 1.48E−07 |
| UniRef90_J7GNA5 | 4.50E−21 | 4.62E−06 | 2.20E−09 |
| UniRef90_J7GFJ7 | 5.05E−21 | 3.67E−06 | 3.47E−09 |
| UniRef90_V3DBN8 | 5.44E−21 | 1.10E−06 | 4.66E−09 |
| UniRef90_A0A012Z9Z8 | 5.51E−21 | 3.65E−06 | 0 |
| UniRef90_A0A015NQF4 | 5.51E−21 | 5.77E−07 | 0 |
| UniRef90_D0TY90 | 5.51E−21 | 8.95E−07 | 0 |
| UniRef90_D7IXV0 | 5.51E−21 | 3.96E−06 | 0 |
| UniRef90_KIRG83 | 5.51E−21 | 1.64E−06 | 0 |
| UniRef90_S2ZSM7 | 5.51E−21 | 4.68E−06 | 0 |
| UniRef90_U6R9J9 | 5.51E−21 | 6.54E−07 | 0 |
| UniRef90_UPI000469370C | 5.51E−21 | 2.53E−06 | 0 |
| UniRef90_J7G851 | 6.06E−21 | 6.56E−06 | 5.81E−08 |
| UniRef90_N6N662 | 7.90E−21 | 3.57E−07 | 6.27E−09 |
| UniRef90_J7GD51 | 7.97E−21 | 4.42E−06 | 1.50E−09 |
| UniRef90_W8YG61 | 8.41E−21 | 2.62E−06 | 8.12E−09 |
| UniRef90_J7GCH9 | 8.45E−21 | 3.55E−06 | 1.74E−09 |
| UniRef90_C3R378 | 8.84E−21 | 1.67E−06 | 2.00E−10 |
| UniRef90_D9RMD1 | 8.84E−21 | 5.76E−06 | 8.25E−10 |
| UniRef90_G5SRF7 | 9.04E−21 | 1.95E−06 | 1.49E−10 |
| UniRef90_Q64WL9 | 9.04E−21 | 6.93E−06 | 3.77E−10 |
| UniRef90_Y8PP51 | 9.04E−21 | 3.17E−06 | 2.83E−10 |
| UniRef90_Q2YTX1 | 9.24E−21 | 6.14E−06 | 1.30E−09 |
| UniRef90_A6QI80 | 9.34E−21 | 4.13E−06 | 7.19E−10 |
| UniRef90_G8LMB5 | 9.53E−21 | 1.14E−06 | 5.74E−08 |
| UniRef90_D5CKJ8 | 9.53E−21 | 1.39E−05 | 2.53E−09 |
| UniRef90_N5ERP1 | 9.66E−21 | 5.43E−07 | 3.37E−10 |
| UniRef90_S3A9Q2 | 9.92E−21 | 7.67E−06 | 4.83E−09 |
| UniRef90_A9CR61 | 1.00E−20 | 6.13E−06 | 8.94E−10 |
| UniRef90_K6A781 | 1.02E−20 | 4.76E−06 | 3.05E−10 |
| UniRef90_Y1F614 | 1.03E−20 | 5.99E−07 | 9.63E−10 |
| UniRef90_J7GCP0 | 1.14E−20 | 4.27E−06 | 1.13E−09 |
| UniRef90_A5IPM0 | 1.27E−20 | 2.41E−06 | 7.48E−08 |
| UniRef90_Y1F410 | 1.48E−20 | 5.10E−06 | 2.73E−09 |
| UniRef90_J7GNK4 | 1.53E−20 | 5.24E−06 | 3.09E−09 |
| UniRef90_J7GJ15 | 1.58E−20 | 4.34E−06 | 2.61E−09 |
| UniRef90_J7GIH1 | 1.66E−20 | 5.19E−06 | 3.68E−09 |
| UniRef90_J7GDL7 | 1.71E−20 | 4.66E−06 | 4.76E−09 |
| UniRef90_L1PR25 | 1.79E−20 | 1.13E−05 | 7.40E−09 |
| UniRef90_J7GKK1 | 1.87E−20 | 3.72E−06 | 2.47E−09 |
| UniRef90_Y1FCB0 | 2.41E−20 | 2.89E−07 | 3.27E−09 |
| UniRef90_W8VES8 | 2.43E−20 | 6.90E−06 | 1.49E−07 |
| UniRef90_J7GBR9 | 2.62E−20 | 9.54E−06 | 3.15E−07 |
| UniRef90_B5XYQ4 | 2.85E−20 | 2.32E−06 | 4.34E−08 |
| UniRef90_J7GBD6 | 2.88E−20 | 5.70E−06 | 2.80E−08 |
| UniRef90_J7GDR2 | 3.47E−20 | 5.97E−06 | 2.90E−08 |
| UniRef90_J7GGF5 | 3.53E−20 | 4.46E−06 | 3.25E−09 |
| UniRef90_B5Y0A0 | 3.70E−20 | 2.33E−06 | 2.59E−08 |
| UniRef90_J7GFL1 | 3.70E−20 | 4.34E−06 | 4.88E−09 |
| UniRef90_D5CE59 | 3.71E−20 | 2.43E−07 | 4.78E−09 |
| UniRef90_UPI00034CA9E6 | 4.23E−20 | 1.03E−05 | 1.26E−07 |
| UniRef90_J7GEI1 | 4.42E−20 | 4.01E−06 | 2.56E−09 |
| UniRef90_C8T071 | 4.55E−20 | 8.98E−07 | 8.30E−09 |
| UniRef90_I0TM81 | 4.94E−20 | 5.73E−06 | 5.10E−08 |
| UniRef90_J7GK50 | 5.02E−20 | 4.64E−06 | 2.79E−09 |
| UniRef90_J7GCZ2 | 5.55E−20 | 4.53E−06 | 2.58E−09 |
| UniRef90_Y1K0I2 | 6.69E−20 | 2.85E−06 | 2.28E−08 |
| UniRef90_J7GJF8 | 8.42E−20 | 4.33E−06 | 2.87E−09 |
| UniRef90_G8LQ28 | 8.87E−20 | 2.34E−06 | 6.45E−08 |
| UniRef90_V3LWH7 | 9.22E−20 | 5.72E−06 | 3.23E−09 |
| UniRef90_J7GHS3 | 9.36E−20 | 3.90E−06 | 3.84E−09 |
| UniRef90_A0A015TXY8 | 9.69E−20 | 3.50E−06 | 0 |
| UniRef90_A0A016KNC5 | 9.69E−20 | 7.90E−07 | 0 |
| UniRef90_B3JEH3 | 9.69E−20 | 8.66E−07 | 0 |
| UniRef90_B5D4M1 | 9.69E−20 | 2.54E−07 | 0 |
| UniRef90_C6ZAN4 | 9.69E−20 | 1.77E−06 | 0 |
| UniRef90_C6ZAP5 | 9.69E−20 | 2.81E−06 | 0 |
| UniRef90_C7XB46 | 9.69E−20 | 1.91E−06 | 0 |
| UniRef90_C9E1D1 | 9.69E−20 | 1.48E−06 | 0 |
| UniRef90_C9KSL6 | 9.69E−20 | 1.10E−06 | 0 |
| UniRef90_D0TY68 | 9.69E−20 | 1.64E−06 | 0 |
| UniRef90_E1Z1I6 | 9.69E−20 | 1.44E−07 | 0 |
| UniRef90_E5UZA7 | 9.69E−20 | 3.81E−07 | 0 |
| UniRef90_G8UJQ4 | 9.69E−20 | 8.53E−08 | 0 |
| UniRef90_K1SCB3 | 9.69E−20 | 8.46E−08 | 0 |
| UniRef90_K1SS36 | 9.69E−20 | 1.85E−06 | 0 |
| UniRef90_K5ZYI4 | 9.69E−20 | 2.44E−06 | 0 |
| UniRef90_Q64WK2 | 9.69E−20 | 4.54E−06 | 0 |
| UniRef90_Q64WK8 | 9.69E−20 | 2.00E−06 | 0 |
| UniRef90_R6A4I6 | 9.69E−20 | 4.03E−07 | 0 |
| UniRef90_S2ZQE6 | 9.69E−20 | 2.46E−06 | 0 |
| UniRef90_T2NFS9 | 9.69E−20 | 7.98E−08 | 0 |
| UniRef90_UPI00046A1900 | 9.69E−20 | 6.55E−07 | 0 |
| UniRef90_W7PD14 | 9.69E−20 | 1.41E−07 | 0 |
| UniRef90_Y8PJ40 | 9.69E−20 | 3.90E−06 | 0 |
| UniRef90_J2ULW3 | 1.02E−19 | 3.22E−07 | 1.27E−08 |
| UniRef90_G8I0W8 | 1.19E−19 | 6.14E−06 | 3.71E−09 |
| UniRef90_G8LIZ8 | 1.29E−19 | 9.68E−06 | 5.58E−07 |
| UniRef90_J7GHB1 | 1.30E−19 | 4.81E−06 | 3.90E−09 |
| UniRef90_J7GBM0 | 1.31E−19 | 3.61E−06 | 4.40E−09 |
| UniRef90_D6SFD4 | 1.34E−19 | 1.24E−05 | 2.03E−07 |
| UniRef90_Y1F344 | 1.39E−19 | 3.09E−06 | 1.08E−06 |
| UniRef90_V3DG42 | 1.40E−19 | 1.84E−06 | 5.86E−08 |
| UniRef90_J7GI93 | 1.47E−19 | 3.87E−06 | 1.27E−08 |
| UniRef90_S4SUQ6 | 1.47E−19 | 1.25E−07 | 1.11E−08 |
| UniRef90_J7GJ06 | 1.53E−19 | 4.10E−06 | 2.32E−09 |
| UniRef90_J7GK44 | 1.54E−19 | 3.58E−06 | 4.18E−09 |
| UniRef90_G8LLP4 | 1.56E−19 | 3.83E−06 | 1.99E−07 |
| UniRef90_A5IR92 | 1.56E−19 | 3.21E−06 | 1.04E−09 |
| UniRef90_C5N3Z5 | 1.56E−19 | 6.44E−06 | 1.34E−09 |
| UniRef90_A0A017N0P3 | 1.57E−19 | 2.47E−06 | 1.30E−10 |
| UniRef90_D7IFP8 | 1.57E−19 | 3.86E−06 | 2.81E−10 |
| UniRef90_F7MCK3 | 1.57E−19 | 1.59E−06 | 5.22E−11 |
| UniRef90_J9GFL6 | 1.57E−19 | 2.54E−06 | 8.14E−11 |
| UniRef90_Y1IW37 | 1.58E−19 | 5.68E−08 | 9.18E−10 |
| UniRef90_C3R3D3 | 1.60E−19 | 5.93E−06 | 1.95E−10 |
| UniRef90_C6Z879 | 1.60E−19 | 2.41E−06 | 2.00E−10 |
| UniRef90_D7IFQ0 | 1.60E−19 | 3.58E−06 | 3.79E−10 |
| UniRef90_E1GVB5 | 1.60E−19 | 1.02E−06 | 4.61E−11 |
| UniRef90_Y1JGA8 | 1.60E−19 | 2.89E−06 | 6.62E−10 |
| UniRef90_J7GDL3 | 1.62E−19 | 4.33E−06 | 3.16E−09 |
| UniRef90_S3ARD4 | 1.62E−19 | 3.44E−06 | 6.42E−09 |
| UniRef90_A0A016NK41 | 1.64E−19 | 8.29E−07 | 2.69E−10 |
| UniRef90_W6EED8 | 1.68E−19 | 1.11E−05 | 4.59E−09 |
| UniRef90_A0A020M651 | 1.72E−19 | 3.82E−06 | 1.67E−09 |
| UniRef90_D1PSS5 | 1.72E−19 | 1.31E−06 | 3.71E−10 |
| UniRef90_UPI00046EE807 | 1.75E−19 | 4.63E−07 | 1.75E−10 |
| UniRef90_E1KW12 | 1.76E−19 | 3.43E−07 | 4.37E−10 |
| UniRef90_W6J8T0 | 1.76E−19 | 3.85E−07 | 1.73E−08 |
| UniRef90_A4W7Q2 | 1.77E−19 | 8.89E−07 | 4.69E−10 |
| UniRef90_B3JID4 | 1.77E−19 | 7.18E−06 | 4.97E−10 |
| UniRef90_C3R372 | 1.79E−19 | 8.93E−06 | 1.24E−08 |
| UniRef90_D7IXQ4 | 1.81E−19 | 1.62E−05 | 4.01E−10 |
| UniRef90_J7GIM2 | 1.82E−19 | 4.68E−06 | 3.27E−09 |
| UniRef90_D7IFQ2 | 1.84E−19 | 3.47E−06 | 4.10E−10 |
| UniRef90_C3R3C3 | 1.87E−19 | 1.63E−05 | 1.90E−08 |
| UniRef90_J7GJU9 | 1.87E−19 | 5.00E−06 | 5.18E−09 |
| UniRef90_C3R376 | 1.94E−19 | 1.35E−05 | 2.17E−08 |
| UniRef90_C3R3C7 | 1.94E−19 | 1.12E−05 | 1.39E−08 |
| UniRef90_K6AYC4 | 2.00E−19 | 2.74E−06 | 4.85E−08 |
| UniRef90_E1KWK7 | 2.05E−19 | 2.61E−07 | 6.21E−10 |
| UniRef90_C3R379 | 2.09E−19 | 7.52E−06 | 1.34E−08 |
| UniRef90_I6S584 | 2.10E−19 | 4.35E−07 | 2.38E−08 |
| UniRef90_S7YUA0 | 2.11E−19 | 5.21E−07 | 5.83E−09 |
| UniRef90_I2FJE4 | 2.13E−19 | 8.50E−06 | 2.87E−08 |
| UniRef90_D7IXF3 | 2.23E−19 | 5.38E−07 | 2.86E−08 |
| UniRef90_D5C5R5 | 2.29E−19 | 4.40E−06 | 4.00E−07 |
| UniRef90_Y1FDD2 | 2.80E−19 | 4.49E−06 | 6.28E−09 |
| UniRef90_J7GCK0 | 2.83E−19 | 5.11E−06 | 1.66E−07 |
| UniRef90_W8UQQ1 | 2.91E−19 | 9.89E−07 | 3.76E−08 |
| UniRef90_J7GNT6 | 2.99E−19 | 4.94E−06 | 3.98E−09 |
| UniRef90_J7GGA1 | 3.06E−19 | 4.53E−06 | 3.31E−09 |
| UniRef90_V3HZ69 | 3.10E−19 | 1.40E−07 | 6.35E−09 |
| UniRef90_J7GIV1 | 3.74E−19 | 4.54E−06 | 1.12E−08 |
| UniRef90_X5G186 | 3.97E−19 | 5.05E−06 | 3.10E−07 |
| UniRef90_J7GFF9 | 4.03E−19 | 4.61E−06 | 2.85E−09 |
| UniRef90_G8LPY0 | 4.06E−19 | 3.69E−06 | 3.66E−07 |
| UniRef90_J7GFS2 | 4.21E−19 | 4.27E−06 | 4.52E−09 |
| UniRef90_J7GJQ3 | 4.24E−19 | 2.54E−06 | 1.36E−08 |
| UniRef90_J7GQN6 | 4.46E−19 | 5.39E−06 | 2.82E−09 |
| UniRef90_J7GK68 | 5.48E−19 | 4.78E−06 | 2.67E−09 |
| UniRef90_W8XJM5 | 5.92E−19 | 3.61E−06 | 7.97E−09 |
| UniRef90_G8LPX0 | 6.63E−19 | 4.64E−08 | 5.21E−08 |
| UniRef90_Y1DBX7 | 6.66E−19 | 2.02E−06 | 2.84E−07 |
| UniRef90_J7GDD2 | 6.95E−19 | 3.60E−06 | 3.16E−09 |
| UniRef90_J7GKF0 | 7.52E−19 | 3.71E−06 | 2.77E−09 |
| UniRef90_G8W1N4 | 7.85E−19 | 2.55E−06 | 4.52E−07 |
| UniRef90_G8LCC0 | 7.98E−19 | 5.72E−07 | 1.96E−08 |
| UniRef90_J7GFP6 | 8.31E−19 | 4.13E−06 | 1.46E−09 |
| UniRef90_Y1JA68 | 8.31E−19 | 1.64E−07 | 3.37E−09 |
| UniRef90_I0JDE4 | 9.41E−19 | 6.83E−07 | 3.38E−09 |
| UniRef90_I4S9D5 | 1.08E−18 | 3.38E−06 | 2.17E−09 |
| UniRef90_J7GCS9 | 1.08E−18 | 3.72E−06 | 3.76E−09 |
| UniRef90_V3ES83 | 1.22E−18 | 8.47E−08 | 5.49E−10 |
| UniRef90_C3R370 | 1.25E−18 | 1.61E−05 | 1.56E−09 |
| UniRef90_J7GCN0 | 1.25E−18 | 8.07E−06 | 2.82E−07 |
| UniRef90_J7GH07 | 1.32E−18 | 5.06E−06 | 2.04E−09 |
| UniRef90_A0A016KE68 | 1.69E−18 | 1.36E−07 | 0 |
| UniRef90_A0A016LR37 | 1.69E−18 | 9.60E−07 | 0 |
| UniRef90_A5IR50 | 1.69E−18 | 2.66E−07 | 0 |
| UniRef90_B5D4L3 | 1.69E−18 | 1.13E−06 | 0 |
| UniRef90_D0TBM8 | 1.69E−18 | 4.57E−07 | 0 |
| UniRef90_D0TYA0 | 1.69E−18 | 7.64E−07 | 0 |
| UniRef90_D1JWS9 | 1.69E−18 | 7.83E−07 | 0 |
| UniRef90_D1JYZ7 | 1.69E−18 | 2.19E−06 | 0 |
| UniRef90_D4VJE1 | 1.69E−18 | 1.31E−06 | 0 |
| UniRef90_D4VS12 | 1.69E−18 | 7.69E−07 | 0 |
| UniRef90_D7IXV1 | 1.69E−18 | 1.39E−06 | 0 |
| UniRef90_E1WRW7 | 1.69E−18 | 2.02E−06 | 0 |
| UniRef90_E5WUL2 | 1.69E−18 | 3.40E−07 | 0 |
| UniRef90_G5SSI1 | 1.69E−18 | 1.17E−06 | 0 |
| UniRef90_I9B632 | 1.69E−18 | 4.32E−07 | 0 |
| UniRef90_J7GIX4 | 1.69E−18 | 1.38E−06 | 0 |
| UniRef90_J9D0V5 | 1.69E−18 | 3.14E−06 | 0 |
| UniRef90_J9G246 | 1.69E−18 | 1.25E−06 | 0 |
| UniRef90_K1T5D9 | 1.69E−18 | 3.05E−07 | 0 |
| UniRef90_Q64WN4 | 1.69E−18 | 6.11E−07 | 0 |
| UniRef90_S0NHM8 | 1.69E−18 | 2.68E−07 | 0 |
| UniRef90_UPI00046A69DB | 1.69E−18 | 7.04E−07 | 0 |
| UniRef90_W8TR24 | 1.69E−18 | 3.26E−07 | 0 |
| UniRef90_X6Q133 | 1.69E−18 | 4.77E−07 | 0 |
| UniRef90_Y1K0M8 | 1.69E−18 | 1.12E−06 | 0 |
| UniRef90_I0TMB7 | 1.70E−18 | 5.49E−06 | 8.76E−08 |
| UniRef90_A7KG22 | 1.72E−18 | 4.15E−06 | 3.61E−08 |
| UniRef90_W8UD91 | 1.76E−18 | 8.83E−07 | 2.06E−08 |
| UniRef90_M7PC36 | 1.83E−18 | 9.61E−06 | 9.47E−10 |
| UniRef90_C3R3D2 | 1.92E−18 | 3.52E−06 | 6.25E−10 |
| UniRef90_W6E2G2 | 1.92E−18 | 6.44E−06 | 1.85E−09 |
| UniRef90_B1RMN0 | 2.07E−18 | 1.18E−05 | 3.85E−09 |
| UniRef90_K4H024 | 2.14E−18 | 3.95E−06 | 7.17E−07 |
| UniRef90_K6AJD4 | 2.23E−18 | 6.45E−06 | 1.14E−09 |
| UniRef90_G8LGR1 | 2.27E−18 | 5.63E−06 | 9.24E−08 |
| UniRef90_N5E8C7 | 2.32E−18 | 6.28E−08 | 1.62E−10 |
| UniRef90_W7P334 | 2.62E−18 | 5.83E−06 | 1.26E−09 |
| UniRef90_C3R385 | 2.67E−18 | 1.32E−05 | 1.23E−09 |
| UniRef90_B1V5I5 | 2.76E−18 | 8.46E−06 | 1.17E−09 |
| UniRef90_C3RFW4 | 2.76E−18 | 3.95E−06 | 5.87E−11 |
| UniRef90_E1WRZ5 | 2.76E−18 | 7.02E−06 | 2.92E−10 |
| UniRef90_F7MCD8 | 2.76E−18 | 2.47E−06 | 4.62E−10 |
| UniRef90_K5ZK81 | 2.76E−18 | 1.27E−06 | 3.02E−10 |
| UniRef90_L6MTF4 | 2.76E−18 | 2.04E−07 | 2.09E−10 |
| UniRef90_R6YDX0 | 2.76E−18 | 9.16E−07 | 6.67E−11 |
| UniRef90_U6R8D0 | 2.76E−18 | 2.49E−06 | 8.83E−11 |
| UniRef90_UPI000403818B | 2.76E−18 | 8.11E−06 | 1.29E−09 |
| UniRef90_Y1IVX0 | 2.76E−18 | 2.92E−06 | 5.73E−10 |
| UniRef90_Y8PQY4 | 2.76E−18 | 4.10E−06 | 7.60E−10 |
| UniRef90_A7X076 | 2.77E−18 | 6.52E−06 | 1.57E−09 |
| UniRef90_A0A016JAH9 | 2.82E−18 | 6.09E−07 | 9.39E−11 |
| UniRef90_D0TY69 | 2.83E−18 | 4.27E−06 | 5.35E−10 |
| UniRef90_A0A016LW33 | 2.88E−18 | 1.08E−06 | 4.07E−10 |
| UniRef90_R5UFY2 | 2.88E−18 | 7.71E−07 | 2.98E−10 |
| UniRef90_C3R3C9 | 2.89E−18 | 1.62E−05 | 7.66E−09 |
| UniRef90_J9G8I9 | 2.95E−18 | 3.45E−07 | 3.63E−10 |
| UniRef90_I0TM71 | 2.95E−18 | 2.52E−05 | 7.69E−07 |
| UniRef90_A7WZU2 | 3.08E−18 | 6.14E−06 | 1.78E−09 |
| UniRef90_C6ZAN2 | 3.08E−18 | 2.97E−06 | 1.68E−10 |
| UniRef90_D0TY31 | 3.08E−18 | 1.59E−06 | 1.32E−10 |
| UniRef90_E1WRZ4 | 3.08E−18 | 7.43E−06 | 6.45E−10 |
| UniRef90_Q64WM9 | 3.08E−18 | 5.80E−06 | 2.96E−10 |
| UniRef90_R5UL26 | 3.08E−18 | 5.09E−06 | 4.62E−10 |
| UniRef90_C3R3D1 | 3.08E−18 | 2.32E−06 | 1.68E−08 |
| UniRef90_U6R9K3 | 3.08E−18 | 1.44E−06 | 1.41E−08 |
| UniRef90_UPI00046CDF83 | 3.12E−18 | 1.42E−05 | 9.13E−09 |
| UniRef90_J7GFM5 | 3.14E−18 | 3.77E−06 | 2.87E−09 |
| UniRef90_J7GHH3 | 3.14E−18 | 5.23E−06 | 2.54E−08 |
| UniRef90_R5DG65 | 3.14E−18 | 3.25E−06 | 2.02E−10 |
| UniRef90_E5UQ60 | 3.15E−18 | 2.54E−06 | 2.69E−08 |
| UniRef90_K5Y4E7 | 3.15E−18 | 2.14E−06 | 7.98E−09 |
| UniRef90_D5CF36 | 3.20E−18 | 9.45E−06 | 1.16E−06 |
| UniRef90_R6EW59 | 3.21E−18 | 6.91E−07 | 7.51E−11 |
| UniRef90_D7IE72 | 3.22E−18 | 8.62E−07 | 1.10E−08 |
| UniRef90_D7IE73 | 3.22E−18 | 2.78E−06 | 2.75E−08 |
| UniRef90_J2X391 | 3.32E−18 | 8.81E−07 | 2.66E−08 |
| UniRef90_Q4ZAM2 | 3.34E−18 | 4.89E−07 | 4.74E−10 |
| UniRef90_Y2YB69 | 3.34E−18 | 7.30E−06 | 5.97E−09 |
| UniRef90_G8LFQ5 | 3.41E−18 | 3.74E−05 | 1.64E−06 |
| UniRef90_K1STW4 | 3.41E−18 | 3.53E−06 | 1.34E−08 |
| UniRef90_C3R0J1 | 3.43E−18 | 1.68E−06 | 3.11E−08 |
| UniRef90_V3RU60 | 3.44E−18 | 5.42E−06 | 5.22E−08 |
| UniRef90_J7G9R2 | 3.48E−18 | 4.46E−06 | 2.40E−09 |
| UniRef90_J7GH01 | 3.48E−18 | 5.81E−06 | 3.27E−09 |
| UniRef90_C3R377 | 3.48E−18 | 9.12E−06 | 1.28E−08 |
| UniRef90_D2EXK8 | 3.48E−18 | 9.03E−07 | 5.88E−09 |
| UniRef90_U2E808 | 3.48E−18 | 1.03E−06 | 1.01E−09 |
| UniRef90_D4IJC0 | 3.51E−18 | 7.01E−06 | 8.95E−08 |
| UniRef90_D7IKR1 | 3.56E−18 | 2.71E−06 | 2.07E−08 |
| UniRef90_Y1F288 | 3.56E−18 | 4.21E−07 | 2.44E−09 |
| UniRef90_D9RMC3 | 3.58E−18 | 5.87E−06 | 1.19E−07 |
| UniRef90_G8LND8 | 3.58E−18 | 5.51E−05 | 1.00E−05 |
| UniRef90_J2X509 | 3.58E−18 | 8.96E−06 | 8.14E−07 |
| UniRef90_J7GL19 | 3.66E−18 | 3.44E−06 | 2.44E−09 |
| UniRef90_UPI00046AE637 | 3.71E−18 | 1.64E−07 | 7.31E−10 |
| UniRef90_J7GEY4 | 3.89E−18 | 5.04E−06 | 3.04E−09 |
| UniRef90_A7KFV6 | 3.96E−18 | 8.04E−06 | 4.24E−08 |
| UniRef90_J7GKQ7 | 3.99E−18 | 3.76E−06 | 1.56E−08 |
| UniRef90_J7GHE8 | 4.01E−18 | 4.02E−06 | 3.99E−09 |
| UniRef90_S2ZS23 | 4.10E−18 | 1.98E−06 | 3.47E−08 |
| UniRef90_G5SRG1 | 4.11E−18 | 5.14E−06 | 2.19E−07 |
| UniRef90_Y8K7A3 | 4.17E−18 | 6.65E−07 | 1.22E−08 |
| UniRef90_J7GI60 | 4.22E−18 | 4.20E−06 | 3.30E−09 |
| UniRef90_W1HYH5 | 4.24E−18 | 2.90E−07 | 9.14E−09 |
| UniRef90_T8JKP3 | 4.28E−18 | 3.26E−06 | 5.23E−08 |
| UniRef90_W8V5D6 | 4.41E−18 | 8.44E−07 | 2.63E−08 |
| UniRef90_J7GI84 | 4.53E−18 | 4.22E−06 | 3.37E−09 |
| UniRef90_D4IN61 | 4.55E−18 | 5.79E−06 | 2.10E−07 |
| UniRef90_J7GF48 | 4.71E−18 | 6.18E−06 | 3.03E−08 |
| UniRef90_Y1K316 | 4.90E−18 | 3.44E−06 | 5.68E−09 |
| UniRef90_M5GV75 | 5.05E−18 | 8.98E−08 | 4.92E−09 |
| UniRef90_J7GE60 | 5.22E−18 | 3.87E−06 | 7.00E−09 |
| UniRef90_F4FNL2 | 5.25E−18 | 6.16E−06 | 4.46E−09 |
| UniRef90_D5CIF5 | 5.41E−18 | 5.60E−06 | 7.05E−07 |
| UniRef90_D5CG96 | 5.67E−18 | 1.21E−05 | 1.01E−06 |
| UniRef90_J7G5S6 | 5.92E−18 | 5.41E−06 | 1.63E−08 |
| UniRef90_J7GHT9 | 6.01E−18 | 5.62E−06 | 1.46E−08 |
| UniRef90_J7GLR1 | 6.21E−18 | 4.91E−06 | 2.16E−09 |
| UniRef90_D5CKD8 | 6.31E−18 | 8.73E−07 | 4.43E−08 |
| UniRef90_J7GEZ4 | 6.32E−18 | 3.64E−06 | 2.02E−09 |
| UniRef90_Y1BGM7 | 6.37E−18 | 6.62E−06 | 1.15E−07 |
| UniRef90_J7GP74 | 6.54E−18 | 5.32E−06 | 2.74E−09 |
| UniRef90_S7TIA6 | 6.76E−18 | 1.96E−06 | 2.78E−07 |
| UniRef90_Y1B3W9 | 6.83E−18 | 5.14E−06 | 3.02E−07 |
| UniRef90_B1RDQ0 | 6.94E−18 | 7.35E−06 | 6.25E−08 |
| UniRef90_J7GI09 | 7.12E−18 | 4.86E−06 | 4.77E−09 |
| UniRef90_W1FRG5 | 7.13E−18 | 3.17E−07 | 2.15E−08 |
| UniRef90_V3I057 | 7.33E−18 | 2.05E−06 | 3.83E−07 |
| UniRef90_W1HRU2 | 7.40E−18 | 2.12E−06 | 4.78E−07 |
| UniRef90_D5CJK2 | 7.66E−18 | 1.06E−05 | 6.74E−07 |
| UniRef90_J7GFY1 | 7.71E−18 | 3.89E−06 | 8.51E−09 |
| UniRef90_J7GEW5 | 8.03E−18 | 5.90E−06 | 2.93E−08 |
| UniRef90_J7GG69 | 8.50E−18 | 6.65E−06 | 8.53E−09 |
| UniRef90_G8LJB7 | 8.62E−18 | 2.67E−06 | 1.56E−07 |
| UniRef90_D5CG31 | 9.04E−18 | 7.35E−08 | 1.25E−09 |
| UniRef90_A4ZFD3 | 9.33E−18 | 5.49E−06 | 2.04E−08 |
| UniRef90_A6QI62 | 9.76E−18 | 2.80E−06 | 7.95E−08 |
| UniRef90_X5G3D0 | 1.04E−17 | 5.51E−06 | 3.39E−07 |
| UniRef90_Q4ZA88 | 1.06E−17 | 1.65E−06 | 5.00E−08 |
| UniRef90_C7ZX47 | 1.08E−17 | 7.37E−06 | 1.96E−08 |
| UniRef90_V3D5A6 | 1.08E−17 | 2.09E−07 | 3.39E−09 |
| UniRef90_D5CJQ5 | 1.08E−17 | 3.02E−06 | 2.43E−07 |
| UniRef90_X5GPS5 | 1.18E−17 | 3.75E−06 | 9.58E−08 |
| UniRef90_D5CDX5 | 1.19E−17 | 1.48E−07 | 3.82E−08 |
| UniRef90_A0A016LWY5 | 1.22E−17 | 1.08E−05 | 2.82E−08 |
| UniRef90_D0K3E6 | 1.25E−17 | 2.06E−06 | 6.31E−08 |
| UniRef90_J7GER0 | 1.26E−17 | 5.67E−06 | 2.45E−09 |
| UniRef90_J7GEM7 | 1.31E−17 | 5.14E−06 | 2.30E−09 |
| UniRef90_W0BTZ6 | 1.35E−17 | 9.12E−06 | 3.74E−07 |
| UniRef90_V3DJD4 | 1.40E−17 | 1.47E−06 | 5.18E−09 |
| UniRef90_Q0P7G4 | 1.41E−17 | 2.88E−06 | 5.99E−07 |
| UniRef90_J7GH48 | 1.49E−17 | 4.47E−06 | 3.89E−09 |
| UniRef90_C3R490 | 1.55E−17 | 3.20E−05 | 2.31E−07 |
| UniRef90_G8LMT6 | 1.58E−17 | 2.49E−06 | 4.19E−08 |
| UniRef90_W1H150 | 1.62E−17 | 1.16E−06 | 7.15E−08 |
| UniRef90_Y1FI85 | 1.65E−17 | 2.72E−06 | 2.60E−08 |
| UniRef90_G2S1U8 | 1.84E−17 | 5.69E−06 | 2.68E−07 |
| UniRef90_W7NUW9 | 1.85E−17 | 5.71E−06 | 3.15E−07 |
| UniRef90_I0TM61 | 1.88E−17 | 1.79E−05 | 1.57E−07 |
| UniRef90_Q8SDT4 | 1.90E−17 | 3.12E−06 | 7.54E−08 |
| UniRef90_J7GJB4 | 1.94E−17 | 3.84E−06 | 2.31E−09 |
| UniRef90_V0IP24 | 1.94E−17 | 1.29E−05 | 5.29E−07 |
| UniRef90_V3Q7L9 | 1.98E−17 | 1.02E−06 | 7.65E−08 |
| UniRef90_J7GB11 | 2.04E−17 | 4.35E−06 | 1.75E−09 |
| UniRef90_C3RFY5 | 2.08E−17 | 1.11E−05 | 1.92E−08 |
| UniRef90_C3R3C5 | 2.11E−17 | 1.44E−05 | 6.83E−09 |
| UniRef90_W1HIJ7 | 2.13E−17 | 1.11E−06 | 7.96E−08 |
| UniRef90_N9UH46 | 2.27E−17 | 3.19E−05 | 1.03E−06 |
| UniRef90_V3EP03 | 2.31E−17 | 6.21E−06 | 6.94E−08 |
| UniRef90_J7GCP6 | 2.35E−17 | 3.97E−06 | 2.06E−09 |
| UniRef90_D2ZIK9 | 2.36E−17 | 4.36E−07 | 1.09E−08 |
| UniRef90_V3SB93 | 2.39E−17 | 2.67E−06 | 9.97E−07 |
| UniRef90_UPI0003A3166C | 2.40E−17 | 1.88E−07 | 5.36E−09 |
| UniRef90_V5B1W0 | 2.44E−17 | 1.37E−07 | 1.48E−08 |
| UniRef90_Q93CC5 | 2.44E−17 | 6.09E−06 | 3.51E−08 |
| UniRef90_UPI0003EB5CD3 | 2.57E−17 | 1.38E−07 | 3.06E−09 |
| UniRef90_D5C6C3 | 2.62E−17 | 4.73E−07 | 7.27E−09 |
| UniRef90_A7KFV4 | 2.69E−17 | 9.78E−06 | 3.69E−08 |
| UniRef90_J7GBY2 | 2.71E−17 | 3.57E−06 | 2.40E−07 |
| UniRef90_V3DBW6 | 2.72E−17 | 7.83E−06 | 1.96E−09 |
| UniRef90_G8LHR5 | 2.77E−17 | 5.04E−06 | 1.81E−07 |
| UniRef90_A0A015P2L2 | 2.93E−17 | 8.85E−08 | 0 |
| UniRef90_C3PZU5 | 2.93E−17 | 3.84E−07 | 0 |
| UniRef90_C6ZAN0 | 2.93E−17 | 2.07E−07 | 0 |
| UniRef90_D0TBM6 | 2.93E−17 | 1.66E−06 | 0 |
| UniRef90_D7IXM8 | 2.93E−17 | 7.35E−07 | 0 |
| UniRef90_E0NQ31 | 2.93E−17 | 3.52E−07 | 0 |
| UniRef90_E5UZB2 | 2.93E−17 | 9.61E−06 | 0 |
| UniRef90_I0PXX7 | 2.93E−17 | 2.39E−07 | 0 |
| UniRef90_J9CPP3 | 2.93E−17 | 1.41E−07 | 0 |
| UniRef90_K1T0Q6 | 2.93E−17 | 1.36E−06 | 0 |
| UniRef90_K1TFR9 | 2.93E−17 | 1.15E−06 | 0 |
| UniRef90_K1TT65 | 2.93E−17 | 5.15E−08 | 0 |
| UniRef90_K1TU74 | 2.93E−17 | 1.54E−06 | 0 |
| UniRef90_K1U8C5 | 2.93E−17 | 5.83E−08 | 0 |
| UniRef90_U2LBR6 | 2.93E−17 | 5.77E−07 | 0 |
| UniRef90_U6R8K6 | 2.93E−17 | 4.25E−07 | 0 |
| UniRef90_UPI0003F937A0 | 2.93E−17 | 1.18E−06 | 0 |
| UniRef90_W1H3X1 | 2.93E−17 | 2.04E−06 | 0 |
| UniRef90_W6NQQ6 | 2.93E−17 | 1.27E−06 | 0 |
| UniRef90_Y1EEX1 | 2.93E−17 | 4.12E−06 | 0 |
| UniRef90_Y1J8T7 | 2.93E−17 | 4.77E−07 | 0 |
| UniRef90_W1G6G6 | 2.95E−17 | 9.09E−07 | 5.80E−08 |
| UniRef90_A0A016LXG0 | 2.99E−17 | 1.56E−05 | 8.23E−10 |
| UniRef90_W8UBV7 | 3.01E−17 | 6.85E−07 | 2.38E−08 |
| UniRef90_Q2YTX0 | 3.04E−17 | 5.17E−06 | 1.22E−09 |
| UniRef90_K5ZSR5 | 3.10E−17 | 1.11E−05 | 9.93E−10 |
| UniRef90_K6BXH8 | 3.10E−17 | 7.45E−06 | 7.00E−10 |
| UniRef90_A0A016JE68 | 3.16E−17 | 1.49E−06 | 5.99E−10 |
| UniRef90_J7GH91 | 3.20E−17 | 6.00E−06 | 2.98E−08 |
| UniRef90_D2Z9D9 | 3.22E−17 | 2.92E−06 | 1.35E−07 |
| UniRef90_D2ZAI4 | 3.38E−17 | 1.05E−06 | 7.12E−08 |
| UniRef90_C3R3A1 | 3.40E−17 | 4.22E−06 | 1.19E−08 |
| UniRef90_C3R3C6 | 3.40E−17 | 1.07E−05 | 2.03E−08 |
| UniRef90_J7GB72 | 3.42E−17 | 3.75E−06 | 9.37E−09 |
| UniRef90_K6BAS4 | 3.47E−17 | 7.18E−06 | 3.48E−09 |
| UniRef90_R5UT23 | 3.47E−17 | 6.38E−06 | 3.70E−08 |
| UniRef90_V3I121 | 3.49E−17 | 4.13E−08 | 6.52E−09 |
| UniRef90_C3R3C4 | 3.53E−17 | 1.37E−05 | 2.83E−08 |
| UniRef90_W7NY15 | 3.59E−17 | 5.74E−06 | 4.13E−07 |
| UniRef90_UPI0003C7A3D4 | 3.60E−17 | 1.92E−05 | 1.70E−06 |
| UniRef90_J7GLW7 | 3.60E−17 | 4.63E−06 | 1.70E−09 |
| UniRef90_Y1JJU8 | 3.60E−17 | 2.24E−06 | 3.70E−07 |
| UniRef90_S3A0M1 | 3.63E−17 | 1.06E−05 | 1.90E−07 |
| UniRef90_S3AUN4 | 3.63E−17 | 1.04E−05 | 1.98E−07 |
| UniRef90_J5ARF9 | 3.67E−17 | 6.00E−06 | 1.53E−07 |
| UniRef90_T0ML71 | 3.74E−17 | 2.38E−07 | 1.92E−09 |
| UniRef90_Q77FU2 | 3.77E−17 | 3.79E−06 | 6.21E−09 |
| UniRef90_L1PTJ0 | 4.03E−17 | 1.53E−05 | 3.24E−07 |
| UniRef90_A6QFY4 | 4.08E−17 | 3.38E−07 | 6.57E−09 |
| UniRef90_A0A016AWE2 | 4.10E−17 | 1.27E−06 | 1.78E−09 |
| UniRef90_W1GNF8 | 4.17E−17 | 1.74E−05 | 1.20E−06 |
| UniRef90_J7GMB5 | 4.22E−17 | 4.58E−06 | 3.34E−09 |
| UniRef90_G8LL41 | 4.33E−17 | 2.80E−07 | 2.22E−08 |
| UniRef90_A0A015XHM2 | 4.40E−17 | 6.09E−06 | 3.83E−10 |
| UniRef90_R6DDL3 | 4.40E−17 | 5.30E−06 | 4.36E−10 |
| UniRef90_A0A015YH34 | 4.49E−17 | 7.23E−06 | 4.60E−10 |
| UniRef90_A0A020QPG9 | 4.49E−17 | 4.56E−06 | 1.39E−09 |
| UniRef90_D1GPR2 | 4.49E−17 | 7.46E−06 | 1.11E−09 |
| UniRef90_K5ZAN0 | 4.49E−17 | 6.05E−06 | 5.99E−10 |
| UniRef90_J7GFR0 | 4.57E−17 | 3.65E−06 | 4.01E−09 |
| UniRef90_D4IN76 | 4.58E−17 | 6.61E−06 | 2.09E−07 |
| UniRef90_A0A016LWS1 | 4.67E−17 | 2.14E−06 | 1.41E−09 |
| UniRef90_Y1B5M2 | 4.72E−17 | 1.28E−06 | 1.66E−07 |
| UniRef90_A0A016CES9 | 4.80E−17 | 3.58E−06 | 1.35E−10 |
| UniRef90_A0A016HD09 | 4.80E−17 | 5.14E−07 | 1.97E−10 |
| UniRef90_B3JIA1 | 4.80E−17 | 6.95E−07 | 5.09E−10 |
| UniRef90_V3RFF5 | 4.80E−17 | 2.63E−06 | 5.13E−10 |
| UniRef90_W7PDX1 | 4.80E−17 | 1.20E−06 | 2.01E−10 |
| UniRef90_C3R3D6 | 4.85E−17 | 9.89E−06 | 7.21E−09 |
| UniRef90_E1WRZ1 | 4.85E−17 | 7.14E−06 | 1.25E−08 |
| UniRef90_J7G150 | 4.85E−17 | 4.46E−06 | 1.71E−09 |
| UniRef90_J7GQF1 | 4.85E−17 | 6.45E−06 | 1.40E−09 |
| UniRef90_A7KFU8 | 4.89E−17 | 2.10E−06 | 1.43E−07 |
| UniRef90_J7GFG9 | 4.91E−17 | 5.22E−06 | 8.81E−10 |
| UniRef90_K1U3H9 | 4.91E−17 | 6.19E−07 | 1.89E−10 |
| UniRef90_K6A129 | 4.91E−17 | 5.03E−07 | 1.67E−10 |
| UniRef90_J7G715 | 4.92E−17 | 5.65E−06 | 1.44E−08 |
| UniRef90_E1XDT5 | 4.94E−17 | 1.80E−07 | 1.01E−08 |
| UniRef90_D6DWL3 | 5.01E−17 | 1.01E−05 | 1.14E−06 |
| UniRef90_V3D766 | 5.03E−17 | 6.56E−08 | 9.84E−10 |
| UniRef90_A0A015TXS0 | 5.04E−17 | 2.90E−06 | 2.47E−08 |
| UniRef90_C3R398 | 5.04E−17 | 1.42E−05 | 7.32E−08 |
| UniRef90_A0A016GGM7 | 5.13E−17 | 6.35E−07 | 3.06E−10 |
| UniRef90_R6ILX6 | 5.13E−17 | 1.06E−06 | 1.33E−09 |

TABLE 3
Most significant KEGG entries for 29-32 cCGA composition identified via
Humann2. Statistical significance is expressed in P-values computed via
Kruskal-Wallis ANOVA. KEGG (Kyoto Encyclopedia of Genes and
Genomes) is a collection of databases dealing with genomes, biological
pathways, diseases, drugs, and chemical substances (Web service URL:
REST see KEGG API). KEGG ID as listed here means K0 entry (namely
an ortholog with same function independently from its taxonomic origin).

| KEGG ID | P-Value | NEC_mean | Preterms_mean |
|---|---|---|---|
| K03427 | 4.79E−23 | 2.11E−06 | 2.54E−08 |
| K07474 | 5.02E−20 | 6.76E−06 | 5.72E−09 |
| K06909 | 2.04E−19 | 9.96E−06 | 8.82E−07 |
| K14059 | 3.18E−16 | 1.27E−05 | 1.91E−07 |
| K13053 | 1.57E−15 | 1.05E−05 | 4.01E−07 |
| K00791 | 8.40E−15 | 5.09E−06 | 1.82E−07 |
| K11040 | 2.17E−13 | 6.09E−06 | 4.58E−08 |
| K02315 | 9.65E−13 | 4.27E−06 | 2.42E−07 |
| K01545 | 1.03E−12 | 5.69E−06 | 3.50E−07 |
| K03559 | 1.42E−12 | 6.27E−06 | 1.05E−09 |
| K13654 | 1.93E−12 | 4.27E−06 | 1.13E−07 |
| K02450 | 2.15E−12 | 5.42E−06 | 4.46E−07 |
| K05606 | 2.20E−12 | 5.79E−06 | 2.18E−07 |
| K02990 | 2.65E−12 | 5.74E−06 | 1.43E−07 |
| K03530 | 2.68E−12 | 1.64E−07 | 1.13E−09 |
| K02342 | 3.34E−12 | 8.29E−06 | 3.44E−08 |
| K02679 | 5.26E−12 | 7.37E−06 | 6.46E−09 |
| K02005 | 9.53E−12 | 5.71E−06 | 1.48E−08 |
| K02956 | 1.03E−11 | 5.22E−06 | 0 |
| K15738 | 1.03E−11 | 4.09E−07 | 0 |
| K03687 | 1.39E−11 | 3.28E−07 | 6.39E−08 |
| K00971 | 1.96E−11 | 9.13E−06 | 2.50E−08 |
| K02426 | 2.13E−11 | 5.42E−06 | 2.20E−07 |
| K00930 | 3.32E−11 | 6.29E−06 | 1.16E−09 |
| K03169 | 6.88E−11 | 1.19E−05 | 2.72E−07 |
| K03215 | 7.46E−11 | 4.56E−06 | 1.21E−07 |
| K11931 | 7.92E−11 | 8.82E−06 | 2.40E−08 |
| K00560 | 9.26E−11 | 2.91E−07 | 0 |
| K02474 | 9.26E−11 | 2.92E−07 | 0 |
| K03190 | 9.47E−11 | 8.46E−08 | 9.83E−08 |
| K03496 | 9.79E−11 | 1.43E−05 | 2.69E−07 |
| K10947 | 1.21E−10 | 5.06E−06 | 1.26E−07 |
| K07313 | 1.21E−10 | 4.61E−06 | 8.50E−08 |
| K11911 | 1.33E−10 | 5.64E−06 | 4.66E−07 |
| K01056 | 1.55E−10 | 4.93E−06 | 1.14E−08 |
| K01818 | 1.62E−10 | 1.11E−06 | 8.40E−11 |
| K03046 | 1.65E−10 | 5.30E−06 | 3.95E−07 |
| K01687 | 1.69E−10 | 1.13E−06 | 2.67E−09 |
| K03791 | 1.84E−10 | 4.34E−07 | 2.90E−09 |
| K01685 | 1.89E−10 | 5.21E−06 | 1.31E−07 |
| K03595 | 1.89E−10 | 5.79E−06 | 1.46E−07 |
| K04656 | 1.96E−10 | 5.79E−07 | 1.72E−08 |
| K02458 | 1.97E−10 | 5.68E−06 | 3.82E−07 |
| K15833 | 2.09E−10 | 8.75E−06 | 6.24E−07 |
| K06180 | 2.33E−10 | 5.90E−06 | 1.56E−07 |
| K07349 | 2.34E−10 | 5.89E−06 | 4.68E−07 |
| K00939 | 2.36E−10 | 5.53E−06 | 1.75E−07 |
| K02032 | 2.43E−10 | 1.06E−08 | 3.20E−10 |
| K07345 | 2.63E−10 | 5.24E−06 | 3.90E−07 |
| K08156 | 2.68E−10 | 1.42E−07 | 1.14E−07 |
| K01704 | 3.01E−10 | 6.06E−06 | 1.30E−07 |
| K02394 | 3.07E−10 | 9.75E−06 | 5.13E−07 |
| K02919 | 3.20E−10 | 2.14E−06 | 2.36E−07 |
| K02079 | 3.64E−10 | 9.97E−06 | 4.41E−07 |
| K03438 | 6.31E−10 | 6.22E−06 | 8.27E−08 |
| K00625 | 6.31E−10 | 5.70E−06 | 1.13E−07 |
| K01613 | 6.73E−10 | 5.73E−06 | 1.37E−07 |
| K17828 | 7.17E−10 | 4.43E−06 | 1.26E−07 |
| K05778 | 7.40E−10 | 2.30E−08 | 6.65E−08 |
| K02065 | 7.59E−10 | 5.55E−06 | 1.49E−07 |
| K06861 | 7.93E−10 | 5.69E−06 | 1.96E−07 |
| K15770 | 8.20E−10 | 2.32E−05 | 2.02E−06 |
| K00831 | 8.30E−10 | 1.28E−07 | 0 |
| K07133 | 8.30E−10 | 5.01E−07 | 0 |
| K02461 | 8.79E−10 | 5.20E−06 | 3.89E−07 |
| K02838 | 9.53E−10 | 5.99E−06 | 2.24E−07 |
| K02680 | 9.92E−10 | 5.81E−06 | 5.26E−07 |
| K14742 | 1.01E−09 | 5.72E−06 | 8.76E−09 |
| K06949 | 1.15E−09 | 5.40E−06 | 1.53E−07 |
| K03657 | 1.19E−09 | 1.34E−05 | 1.60E−06 |
| K12290 | 1.23E−09 | 7.20E−06 | 5.12E−07 |
| K02914 | 1.25E−09 | 8.49E−08 | 1.14E−09 |
| K00175 | 1.30E−09 | 5.54E−06 | 1.44E−07 |
| K10012 | 1.35E−09 | 2.86E−05 | 6.01E−06 |
| K00940 | 1.44E−09 | 6.38E−06 | 1.87E−07 |
| K02775 | 1.47E−09 | 1.21E−06 | 2.28E−10 |
| K02004 | 1.50E−09 | 4.60E−07 | 4.94E−10 |
| K08998 | 1.50E−09 | 6.61E−08 | 1.80E−10 |
| K14989 | 1.50E−09 | 5.62E−07 | 1.14E−09 |
| K00860 | 1.52E−09 | 4.37E−06 | 4.30E−10 |
| K01153 | 1.60E−09 | 2.11E−06 | 3.31E−08 |
| K08680 | 1.64E−09 | 7.09E−08 | 7.93E−08 |
| K11991 | 1.67E−09 | 5.78E−06 | 3.50E−07 |
| K02622 | 1.69E−09 | 2.43E−06 | 2.96E−09 |
| K07154 | 1.76E−09 | 8.85E−06 | 6.43E−07 |
| K06907 | 1.84E−09 | 6.43E−06 | 6.79E−07 |
| K02083 | 1.87E−09 | 7.68E−06 | 5.52E−07 |
| K01752 | 1.98E−09 | 5.43E−06 | 1.24E−07 |
| K02473 | 2.05E−09 | 1.17E−06 | 6.64E−08 |
| K07644 | 2.12E−09 | 3.26E−06 | 3.69E−07 |
| K00790 | 2.15E−09 | 5.80E−06 | 1.37E−07 |
| K00041 | 2.21E−09 | 4.78E−06 | 1.19E−07 |
| K00812 | 2.27E−09 | 5.78E−06 | 1.49E−07 |
| K00979 | 2.28E−09 | 4.54E−06 | 8.14E−09 |
| K00854 | 2.33E−09 | 4.70E−06 | 1.18E−07 |
| K01629 | 2.33E−09 | 6.02E−06 | 1.53E−07 |
| K04567 | 2.50E−09 | 5.69E−06 | 1.33E−07 |
| K04763 | 2.62E−09 | 2.81E−06 | 6.08E−09 |
| K00765 | 2.82E−09 | 5.24E−06 | 2.80E−07 |
| K00826 | 2.86E−09 | 6.07E−06 | 2.25E−07 |
| K01674 | 3.22E−09 | 6.20E−06 | 4.66E−07 |
| K15586 | 3.37E−09 | 3.73E−06 | 8.65E−07 |
| K02907 | 3.42E−09 | 9.95E−06 | 2.23E−06 |
| K01951 | 3.56E−09 | 5.32E−06 | 3.26E−08 |
| K01247 | 3.90E−09 | 2.44E−08 | 7.75E−08 |
| K15634 | 4.00E−09 | 8.10E−06 | 3.71E−09 |
| K02916 | 4.45E−09 | 6.39E−06 | 1.85E−07 |
| K02895 | 4.52E−09 | 6.19E−06 | 2.17E−07 |
| K06041 | 4.52E−09 | 6.52E−06 | 1.60E−07 |
| K02437 | 4.52E−09 | 5.77E−06 | 1.70E−07 |
| K01810 | 4.71E−09 | 5.65E−06 | 1.36E−07 |
| K02876 | 4.98E−09 | 5.44E−06 | 1.79E−07 |
| K04757 | 4.99E−09 | 4.95E−06 | 4.15E−07 |
| K02038 | 5.12E−09 | 5.66E−06 | 2.37E−07 |
| K07107 | 5.12E−09 | 6.43E−06 | 1.79E−07 |
| K07560 | 5.27E−09 | 6.39E−06 | 1.82E−07 |
| K02879 | 5.34E−09 | 5.73E−06 | 2.10E−07 |
| K00793 | 5.56E−09 | 1.28E−07 | 1.52E−08 |
| K06904 | 5.64E−09 | 6.17E−06 | 3.58E−08 |
| K14652 | 6.02E−09 | 5.84E−06 | 1.36E−07 |
| K02074 | 6.05E−09 | 8.51E−06 | 7.41E−09 |
| K03147 | 6.26E−09 | 5.05E−06 | 1.26E−07 |
| K00626 | 6.54E−09 | 5.12E−06 | 4.17E−07 |
| K01885 | 7.22E−09 | 6.06E−06 | 1.99E−07 |
| K06942 | 7.22E−09 | 6.50E−06 | 1.93E−07 |
| K19048 | 7.61E−09 | 6.19E−08 | 1.19E−07 |
| K00648 | 7.71E−09 | 5.88E−06 | 2.08E−07 |
| K03522 | 8.33E−09 | 5.73E−06 | 1.91E−07 |
| K02902 | 8.54E−09 | 5.37E−06 | 9.89E−07 |
| K01462 | 9.05E−09 | 4.86E−06 | 1.30E−07 |
| K03664 | 9.18E−09 | 4.99E−06 | 1.29E−07 |
| K09810 | 1.01E−08 | 6.00E−06 | 1.54E−07 |
| K12410 | 1.06E−08 | 6.07E−06 | 1.63E−07 |
| K04335 | 1.19E−08 | 6.17E−06 | 4.90E−07 |
| K03817 | 1.33E−08 | 3.59E−06 | 3.39E−07 |
| K03088 | 1.39E−08 | 1.07E−05 | 2.90E−07 |
| K01838 | 1.78E−08 | 4.99E−06 | 4.43E−07 |
| K03563 | 1.82E−08 | 6.38E−08 | 1.16E−08 |
| K09824 | 1.91E−08 | 5.46E−06 | 4.43E−07 |
| K06957 | 1.99E−08 | 5.49E−06 | 4.25E−07 |
| K01821 | 2.45E−08 | 4.73E−06 | 2.82E−07 |
| K02341 | 2.69E−08 | 1.56E−07 | 1.49E−07 |
| K19302 | 2.70E−08 | 1.66E−05 | 2.33E−07 |
| K06905 | 2.86E−08 | 6.23E−06 | 4.91E−07 |
| K01066 | 4.87E−08 | 6.50E−06 | 6.42E−07 |
| K03386 | 6.02E−08 | 3.07E−06 | 8.85E−07 |
| K03764 | 8.77E−08 | 3.03E−07 | 1.51E−07 |
| K00839 | 8.89E−08 | 9.57E−08 | 1.08E−07 |
| K06155 | 9.99E−08 | 9.71E−06 | 1.76E−06 |
| K15722 | 1.12E−07 | 5.57E−06 | 1.30E−06 |
| K01265 | 1.36E−07 | 1.70E−06 | 3.26E−07 |
| K00850 | 2.79E−07 | 2.70E−05 | 8.46E−06 |
| K08225 | 3.63E−07 | 3.72E−06 | 1.67E−06 |
| K13408 | 5.03E−07 | 8.13E−06 | 9.97E−06 |
| K01892 | 7.44E−07 | 1.56E−07 | 9.33E−08 |

In a further analysis, the top 100 predictive stratified superpathways were identified from the gini feature importances of trained models (Table 4). The index of each ranked feature was taken for each model and compared across models. This demonstrates the process for developing new biomarkers based on AI models.

TABLE 4
Top 100 predictive stratified superpathways identified from the gini
importances of trained models. Harmonic Mean of Index is comparing the agreeance of
important features between 8 different models by ordering features in order of
descending gini importance and calculating the harmonic mean of the resulting index
location for each feature.

| | Harmonic Mean |
|---|---|
| Feature | of Index |
| PWY-7328: superpathway of UDP-glucose-derived O-antigen | [1.98731185] |
| building blocks biosynthesis\|g_Escherichia.s_Escherichia_coli | |
| RHAMCAT-PWY: L-rhamnose degradation | [4.18225749] |
| I\|g_Enterococcus.s_Enterococcus_faecalis | |
| AST-PWY: L-arginine degradation II (AST | [4.3726274] |
| pathway\|g_Citrobacter.s_Citrobacter_freundii | |
| PWY-6467: Kdo transfer to lipid IVA III | [4.64789805] |
| (Chlamydia)\|g_Escherichia.s_Escherichia_coli | |
| PWY-6708: ubiquinol-8 biosynthesis | [4.80645861] |
| (prokaryotic)\|g_Enterobacter.s_Enterobacter_cloacae | |
| PWY-7111: pyruvate fermentation to isobutanol | [6.61434857] |
| (engineered)\|g_Klebsiella.s_Klebsiella_oxytoca | |
| ARGININE-SYN4-PWY: L-ornithine de novo biosynthesis\|unclassified | [7.18514698] |
| OANTIGEN-PWY: O-antigen building blocks biosynthesis | [9.51092692] |
| (E. coli)\|g_Escherichia.s_Escherichia_coli | |
| DTDPRHAMSYN-PWY: dTDP-L-rhamnose biosynthesis | [9.78417266] |
| I\|g_Veillonella.s_Veillonella_atypica | |
| PWY-4981: L-proline biosynthesis II (from | [9.83505858] |
| arginine)\|g_Escherichia.s_Escherichia_coli | |
| KETOGLUCONMET-PWY: ketogluconate | [10.68115979] |
| metabolism\|g_Escherichia.s_Escherichia_coli | |
| PWY-7219: adenosine ribonucleotides de novo | [11.38770062] |
| biosynthesis\|g_Peptostreptococcaceae_noname.s_Clostridium_difficile | |
| UNINTEGRATED\|g_Mycoplasma.s_Mycoplasma_hominis | [11.48942509] |
| FASYN-INITIAL-PWY: superpathway of fatty acid biosynthesis initiation | [11.66999399] |
| (E. coli)\|g_Haemophilus.s_Haemophilus_parainfluenzae | |
| NAD-BIOSYNTHESIS-II: NAD salvage pathway | [11.90630957] |
| II\|g_Klebsiella.s_Klebsiella_pneumoniae | |
| PWY-6123: inosine-5′-phosphate biosynthesis | [12.35825811] |
| I\|g_Staphylococcus.s_Staphylococcus_epidermidis | |
| PWY-5855: ubiquinol-7 biosynthesis | [13.4933299] |
| (prokaryotic)\|g_Enterobacter.s_Enterobacter_cloacae | |
| PWY0-1241: ADP-L-glycero-&beta;-D-manno-heptose | [13.81267772] |
| biosynthesis\|g_Enterobacter.s_Enterobacter_cloacae | |
| PWY-6519: 8-amino-7-oxononanoate biosynthesis | [15.15546846] |
| I\|g_Enterobacter.s_Enterobacter_cloacae | |
| PANTO-PWY: phosphopantothenate biosynthesis | [15.77075078] |
| I\|g_Enterococcus.s_Enterococcus_faecalis | |
| PWY-5347: superpathway of L-methionine biosynthesis | [16.43749869] |
| (transsulfuration)\|g_Escherichia.s_Escherichia_coli | |
| PWY-5989: stearate biosynthesis II (bacteria and | [16.66042309] |
| plants)\|g_Enterobacter.s_Enterobacter_cloacae | |
| PWY-6121: 5-aminoimidazole ribonucleotide biosynthesis | [16.84799827] |
| I\|g_Haemophilus.s_Haemophilus_parainfluenzae | |
| UDPNAGSYN-PWY: UDP-N-acetyl-D-glucosamine biosynthesis | [17.63145848] |
| I\|g_Peptostreptococcaceae_noname.s_Clostridium_difficile | |
| PWY-6147: 6-hydroxymethyl-dihydropterin diphosphate biosynthesis | [17.98127811] |
| I\|g_Enterobacter.s_Enterobacter_cloacae | |
| VALSYN-PWY: L-valine | [19.6166336] |
| biosynthesis\|g_Peptostreptococcaceae_noname.s_Clostridium_difficile | |
| PWY-5856: ubiquinol-9 biosynthesis | [20.72875672] |
| (prokaryotic)\|g_Enterobacter.s_Enterobacter_cloacae | |
| PWY-5173: superpathway of acetyl-CoA | [21.05056623] |
| biosynthesis\|g_Escherichia.s_Escherichia_coli | |
| PWY-5138: unsaturated, even numbered fatty acid &beta,- | [23.24598732] |
| oxidation\|g_Citrobacter.s_Citrobacter_freundii | |
| PWY-724: superpathway of L-lysine, L-threonine and L-methionine | [23.2994216] |
| biosynthesis II\|unclassified | |
| LPSSYN-PWY: superpathway of lipopolysaccharide | [23.85251905] |
| biosynthesis\|g_Escherichia.s_Escherichia_coli | |
| UNINTEGRATED\|g_Klebsiella.s_Klebsiella_oxytoca | [24.33554125] |
| PWY-6731: starch degradation III\|g_Klebsiella.s_Klebsiella_oxytoca | [24.65831496] |
| PWY-5384: sucrose degradation IV (sucrose | [24.97046729] |
| phosphorylase)\|g_Escherichia.s_Escherichia_coli | |
| PWY-7219: adenosine ribonucleotides de novo | [25.40783623] |
| biosynthesis\|g_Enterococcus.s_Enterococcus_faecalis | |
| UNINTEGRATED\|g_Enterococcus.s_Enterococcus_faecalis | [25.68700532] |
| PWY-5022: 4-aminobutanoate degradation | [26.22829055] |
| V\|g_Klebsiella.s_Klebsiella_pneumoniae | |
| ILEUSYN-PWY: L-isoleucine biosynthesis I (from | [26.55317053] |
| threonine)\|g_Enterobacter.s_Enterobacter_cloacae | |
| PWY-6387: UDP-N-acetylmuramoyl-pentapeptide biosynthesis I (meso- | [26.77992529] |
| diaminopimelate containing)\|g_Enterobacter.s_Enterobacter_cloacae | |
| PWY-6277: superpathway of 5-aminoimidazole ribonucleotide | [27.07792208] |
| biosynthesis\|g_Campylobacter.s_Campylobacter_ureolyticus | |
| PWY-5686: UMP biosynthesis\|g_Enterobacter.s_Enterobacter_aerogenes | [27.89682396] |
| PWY-7198: pyrimidine deoxyribonucleotides de novo biosynthesis | [28.60099256] |
| IV\|g_Haemophilus.s_Haemophilus_parainfluenzae | |
| PWY-5347: superpathway of L-methionine biosynthesis | [29.29932665] |
| (transsulfuration)\|g_Klebsiella.s_Klebsiella_oxytoca | |
| PWY-6122: 5-aminoimidazole ribonucleotide bio synthesis | [30.17851735] |
| II\|g_Enterococcus.s_Enterococcus_faecalis | |
| THRESYN-PWY: superpathway of L-threonine | [30.5083275] |
| biosynthesis\|g_Haemophilus.s_Haemophilus_parainfluenzae | |
| HISTSYN-PWY: L-histidine | [31.36604867] |
| biosynthesis\|g_Staphylococcus.s_Staphylococcus_epidermidis | |
| PANTO-PWY: phosphopantothenate biosynthesis | [32.59355886] |
| I\|g_Klebsiella.s_Klebsiella_oxytoca | |
| UNINTEGRATED\|g_Propionibacterium.s_Propionibacterium_avidum | [32.69121946] |
| HISDEG-PWY: L-histidine degradation | [34.79045578] |
| I\|g_Enterobacter.s_Enterobacter_cloacae | |
| METSYN-PWY: L-homoserine and L-methionine | [35.51236308] |
| biosynthesis\|g_Escherichia.s_Escherichia_coli | |
| PWY0-1586: peptidoglycan maturation (meso-diaminopimelate | [35.75156772] |
| containing)\|g_Klebsiella.s_Klebsiella_oxytoca | |
| HEMESYN2-PWY: heme biosynthesis II | [36.45206441] |
| (anaerobic)\|g_Escherichia.s_Escherichia_coli | |
| PWY0-1298: superpathway of pyrimidine deoxyribonucleosides | [37.59722142] |
| degradation\|g_Enterobacter.s_Enterobacter_cloacae | |
| TRPSYN-PWY: L-tryptophan | [38.95818071] |
| biosynthesis\|g_Staphylococcus.s_Staphylococcus_aureus | |
| UNINTEGRATED\|g_Caulobacter.s_Caulobacter_vibrioides | [39.08475347] |
| PWY-5189: tetrapyrrole biosynthesis II (from | [40.30714443] |
| glycine)\|g_Staphylococcus.s_Staphylococcus_epidermidis | |
| PWY-7219: adenosine ribonucleotides de novo | [40.51649759] |
| biosynthesis\|g_Bifidobacterium.s_Bifidobacterium_bifidum | |
| PWY-2941: L-lysine biosynthesis | [40.89604138] |
| II\|g_Enterococcus.s_Enterococcus_faecalis | |
| PWY-7357: thiamin formation from pyrithiamine and oxythiamine | [41.81486754] |
| (yeast)\|g_Klebsiella.s_Klebsiella_pneumoniae | |
| PWY-7039: phosphatidate metabolism, as a signaling | [41.93685967] |
| molecule\|g_Escherichia.s_Escherichia_coli | |
| GLYOXYLATE-BYPASS: glyoxylate | [41.94540028] |
| cycle\|g_Enterobacter.s_Enterobacter_cloacae | |
| PWY-7219: adenosine ribonucleotides de novo | [42.70793053] |
| biosynthesis\|g_Propionibacterium.s_Propionibacterium_avidum | |
| PWY66-422: D-galactose degradation V (Leloir | [42.742751] |
| pathway)\|g_Escherichia.s_Escherichia_coli | |
| PWY66-389: phytol de gradation\|g_Klebsiella.s_Klebsiella_pneumoniae | [43.21112134] |
| PWY-6277: superpathway of 5-aminoimidazole ribonucleotide | [44.40830275] |
| biosynthesis\|g_Enterococcus.s_Enterococcus_faecalis | |
| PWY-6901: superpathway of glucose and xylose | [44.71833113] |
| degradation\|g_Enterobacter.s_Enterobacter_cloacae | |
| LACTOSECAT-PWY: lactose and galactose degradation | [44.78218139] |
| I\|g_Enterococcus.s_Enterococcus_faecalis | |
| COA-PWY-1: coenzyme A biosynthesis II | [45.37127998] |
| (mammalian)\|g_Enterococcus.s_Enterococcus_faecalis | |
| GOLPDLCAT-PWY: superpathway of glycerol degradation to 1,3- | [46.00392444] |
| propanediol\|g_Escherichia.s_Escherichia_coli | |
| BIOTIN-BIOSYNTHESIS-PWY: biotin biosynthesis | [46.39372633] |
| I\|g_Enterobacter.s_Enterobacter_cloacae | |
| UNINTEGRATED\|g_Staphylococcus.s_Staphylococcus_epidermidis | [46.78802693] |
| PWY-6163: chorismate biosynthesis from 3- | [46.84240696] |
| dehydroquinate\|g_Staphylococcus.s_Staphylococcus_epidermidis | |
| PWY-7234: inosine-5\|-phosphate biosynthesis | [47.28562194] |
| III\|g_Streptococcus.s_Streptococcus_agalactiae | |
| PWY-6121: 5-aminoimidazole ribonucleotide biosynthesis | [48.66389169] |
| I\|g_Enterococcus.s_Enterococcus_faecalis | |
| PWY0-1586: peptidoglycan maturation (meso-diaminopimelate | [48.84533758] |
| containing)\|g_Enterobacter.s_Enterobacter_aerogenes | |
| UNINTEGRATED\|unclassified | [50.3823845] |
| BRANCHED-CHAIN-AA-SYN-PWY: superpathway of branched | [51.43046101] |
| amino acid biosynthesis\|unclassified | |
| PWY0-1319: CDP-diacylglycerol biosynthesis | [52.28442671] |
| II\|g_Haemophilus.s_Haemophilus_parainfluenzae | |
| PWY-6277: superpathway of 5-aminoimidazole ribonucleotide | [52.49950057] |
| biosynthesis\|g_Haemophilus.s_Haemophilus_parainfluenzae | |
| TRPSYN-PWY: L-tryptophan | [53.0112067] |
| biosynthesis\|g_Staphylococcus.s_Staphylococcus_epidermidis | |
| PWY-6126: superpathway of adenosine nucleotides de novo biosynthesis | [53.81218944] |
| II\|g_Haemophilus.s_Haemophilus_parainfluenzae | |
| HEME−BIOSYNTHESIS-II: heme biosynthesis I | [54.2885475] |
| (aerobic)\|g_Staphylococcus.s_Staphylococcus_epidermidis | |
| ASPASN-PWY: superpathway of L-aspartate and L-asparagine | [57.09381494] |
| biosynthesis\|g_Haemophilus.s_Haemophilus_parainfluenzae | |
| PANTO-PWY: phosphopantothenate biosynthesis | [57.6415431] |
| I\|g_Peptostreptococcaceae_noname.s_Clostridium_difficile | |
| PWY-7220: adenosine deoxyribonucleotides de novo biosynthesis | [57.81640106] |
| II\|unclassified | |
| UNINTEGRATED\|g_Peptostreptococcaceae_noname.s_Clostridium_ | [58.30836637] |
| sordellii | |
| PWY-5857: ubiquinol-10 biosynthesis | [60.91242848] |
| (prokaryotic)\|g_Enterobacter.s_Enterobacter_cloacae | |
| AEROBACTINSYN-PWY: aerobactin | [61.42418831] |
| biosynthesis\|g_Escherichia.s_Escherichia_coli | |
| P164-PWY: purine nucleobases degradation I | [61.64646273] |
| (anaerobic)\|g_Peptostreptococcaceae_noname.s_Clostridium_difficile | |
| HOMOSER-METSYN-PWY: L-methionine biosynthesis | [61.70485371] |
| I\|g_Klebsiella.s_Klebsiella_oxytoca | |
| PWY-5100: pyruvate fermentation to acetate and lactate | [61.84176904] |
| II\|g_Enterococcus.s_Enterococcus_faecalis | |
| TCA: TCA cycle I (prokaryotic)\|g_Klebsiella.s_Klebsiella_oxytoca | [62.21570994] |
| UNINTEGRATED\|g_Haemophilus.s_Haemophilus_parainfluenzae | [62.38327058] |
| PWY-7388: octanoy-[acyl-carrier protein] biosynthesis (mitochondria, | [62.96015905] |
| yeast)\|unclassified | |
| PWY-6606: guanosine nucleotides degradation | [63.40356957] |
| II\|g_Escherichia.s_Escherichia_coli | |
| UNINTEGRATED\|g_Escherichia.s_Escherichia_coli | [63.47892485] |
| PWY-5667: CDP-diacylglycerol biosynthesis | [65.45618728] |
| I\|g_Haemophilus.s_Haemophilus_parainfluenzae | |
| PWY-7221: guanosine ribonucleotides de novo | [65.5033973] |
| biosynthesis\|g_Enterococcus.s_Enterococcus_faecalis | |
| COA-PWY-1: coenzyme A biosynthesis II | [65.86654128] |
| (mammalian)\|g_Streptococcus.s_Streptococcus_agalactiae | |
| PWY0-1061: superpathway of L-alanine | [66.24709326] |
| biosynthesis\|g_Escherichia.s_Escherichia_coli | |

Protein and superpathway Identified among samples. The largest dataset produced represented a matrix of 11,026,566 (Uniref90 hits)×1,605 (samples; 245 NEC positive) or 17.7 billion entries. Gene family entries were converted into pathways. By default, HUMAnN2 uses MetaCyc pathway definitions and MinPath to identify a parsimonious set of pathways that explain observed reactions in the community. This led to a matrix of 1,605 (samples)×595 (pathway) or ˜955 thousand entries. The stratified matrix had 18,442 features when considering the superpathway and the respective contributing bacterial species. First, we used Principal Component Analysis (PCA) to investigate our data set across both taxonomic and gene features. This revealed insights into the structure of the data from both a sample and a feature perspective. Second, we divided the sampling size into different subsets based on corrected gestational age and applied random forest techniques to assess whether the NEC or healthy preterm status could be predicted based on microbiome signatures. Since there is no previous indication on which microbial feature should be over or under abundant in NEC vs. healthy preterm state, we used the Kruskal-Wallis test coupled with Bonferroni correction to determine the subset of gene families that are most statistically significant between NEC and healthy preterms. From the Kruskal-Wallis test we selected entries with an adjusted p<0.0001 (Bonferroni). The 3,420 significant gene families were then converted into KEGG functional orthologs (KO), resulting in 155 KO features. Therefore, we have determined the most statistically significant over and under abundant KEGGs in NEC state.

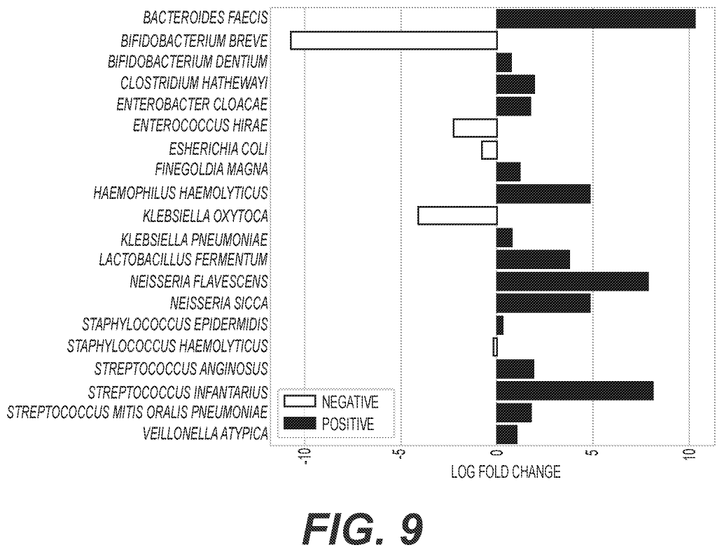

Microbial-driven arginine depletion in the Intestine is characteristic of NEC. 2,732 biomarkers presented the highest risk for NEC from a combination of KEGG ID with a specific bacterial species. When grouping those biomarkers by the pathway they are involved in, we identified among those, the Microbiome-mediated arginine (Arg) metabolism pathway, to be different in the NEC cases compared to controls (FIG. 8). In FIG. 8, EC 2.6.1.1 (Acetylornithine transaminase) and EC 3.5.1.5 (urease) had highest gene abundance (***)relative to the preterm controls whereas 3.5.1.2 (glutaminase) and 1.4.1.3 (glutamate dehydrogenase) were several folds lower (#) in the NEC samples compared to the preterm controls. EC 1.4.1.4 (glutamate dehydrogenase), 2.1.3.3 (ornithine carbamoyltransferase); 2.6.1.11 (acetylornithine aminotransferase); EC3.5.3.6 (arginine deaminase); 2.3.1.1 (amino-acid N-acetyltransferase); 2.7.2.8 (acetylglutamate kinase) were the next highest gene abundance (**(in NEC vs Control, then the group 2.6.1.2; 6.3.1.2; 2.7.2.2 (carbamate kinase) and 6.3.4.5 (arginosuccinate) were still significantly higher (*) in NEC vs. preterm control. Multiple key genes involved in the Arg pathway were several fold higher in the NEC samples compared to preterm controls. Systemic Arg depletion has been reported in NEC. Arg substrate are diverted from secondary pathways, particularity nitric oxide (NO), a critical mediator of vasodilation, blood flow and tissue oxygenation (Reaction KEGG ID: R11711, R11712, R11713). Specific bacterial species were responsible for the arginine pathway depletion (FIG. 9). Particularly, the absence of key beneficial bacteria such as bifidobacteria in the NEC cohort, in conjunction with higher level of potentially pathogenic bacteria (signature of dysbiosis), could lead to arginine depletion as a mechanism of virulence enabling host immune evasion. Neonatal pathogens Streptcooccus sp. and Klebsiella sp. are known to increase production of ornithine, indicating a strong shift in the arginine deiminase pathway activity, resulting in limited Arg availability for NO synthesis due to substrate deprivation for nitric oxide synthases (NOS, KEGG ID: 1.14.14.47; Reaction KEGG ID: R11711, R11712, R11713).

TABLE 5
The most important genes that distinguish NEC from control preterm infants

| | | | | | | Healthy | | Log2 | |
|---|---|---|---|---|---|---|---|---|---|
| | | | | | | preterm | NEC | FC | Fold |
| ID | Protein names | Gene names | Organism | Length | ID_proc | mean | mean | (NEC) | Change |
| G8LMZ9_ENTCL | Acid shock | asr | Enterobacter cloacae | 131 | UniRef90_G8LMZ9 | 4.96082E−08 | 3.92117E−07 | 2.982632526 | 17.1723448 |
| | protein | EcWSU1_01978 | EcWSU1 | | | | | | |
| E11414_9 | Addiction module | HMPREF9321_0318 | Veillonella atypica | 87 | UniRef90_E1L414 | 1.58924E−06 | 1.96384E−05 | 3.627270921 | 15.96957 |
| | toxin, RelE/ | | ACS-049-V-Sch [symbol] | | | | | | |
| | StbE family | | | | | | | | |
| W1DIL6_KLEPN | Adenosyl | | Klebsiella pneumoniae IS43 | 51 | UniRef90_W1DIL6 | 6.31497E−08 | 8.97591E−07 | 3.82921067 | 12.4392233 |
| | homocysteinase | | | | | | | | |
| | (EC 3.3.1.1) | | | | | | | | |
| W9BPS5_KLEPN | AraC family | BN49_3660 | Klebsiella pneumoniae | 268 | UniRef90_W9BPS5 | 1.00645E−06 | 1.03769E−05 | 3.366021626 | 9.15656849 |
| | transcriptional | D0897_02260 | | | | | | | |
| | regulator | | | | | | | | |
| X8H364_9FIRM | Arylsufatase | HMPREF1504_0052 | Veillonella sp. ICM51a | 672 | UniRef90_X8H364 | 5.06288E−07 | 1.5793E−05 | 4.963187371 | 29.206341 |
| | (EC 3.1.6.—) | | | | | | | | |
| D6D3M7_9BACE | ATPases involved in | [symbol] XY_41090 | Bacteroides xylanisolvens | 260 | UniRef90_D6D3M7 | 9.38901E−10 | 7.7945E−06 | 13.01919659 | 25655.6179 |
| | chromosome | | XB1A | | | | | | |
| | partitioning | | | | | | | | |
| G2S602_ENTAL | Cell division | sulA Entas_1463 | Enterobacter asburiae | 187 | UniRef90_G2S602 | 3.06812E−07 | 1.05444E−05 | 5.102975808 | 28.5058564 |
| | inhibitor SulA | | (strain LF7a [symbol] | | | | | | |
| A0A017N0P3_BACFG | CcbQ/CcbB/MinD/Par | M138_4625 | Bacteroides fragilis str. | 251 | UniRef90_A0A017N0P3 | 1.2981E−10 | 2.46706E−06 | 14.21410485 | inf |
| | Anucleotide | M138_4744 | S23L17 | | | | | | |
| | binding do [symbol] | | | | | | | | |
| G8LJG5_ENTCL | Cytochrome | [symbol] ceJ | Enterobacter cloacae EcWSU1 | 194 | UniRef90_G8LJG5 | 4.39623E−07 | 9.95359E−06 | 4.500877515 | 24.2893728 |
| | b561-like | EcWSU1_01646 | | | | | | | |
| | protein 2 | | | | | | | | |
| A7KFV8_KLEPN | HipA (HipA | hipA | Klebsiella pneumoniae | 441 | UniRef90_A7KFV8 | 4.91868E−07 | 8.85215E−06 | 4.169685376 | 13.9173474 |
| | protein) | SAMEA4394728_04998 | | | | | | | |
| | (EC 2.7.11.1) | | | | | | | | |
| C3RFZ0_9BACE | HipA-like C-terminal | BSEG_04090 | Bacteroides dorei 5_1_36/D4 | 529 | UniRef90_C3RFZ0 | 5.62156E−08 | 1.04309E−05 | 7.535675924 | 214.529298 |
| | domain protein | | | | | | | | |
| A0A015XHM2_BACFG | HipA-like | M136_5131 | Bacteroides fragilis str. | 300 | UniRef90_A0A015XHM2 | 3.82875E−10 | 6.09365E−06 | 13.95814402 | 15813.1991 |
| | N-terminal | | S36L11 | | | | | | |
| | domain protein | | | | | | | | |
| W9BAX7_KLEPN | HlyD family | BN49_3658 | Klebsiella pneumoniae | 287 | UniRef90_W9BAX7 | 1.02764E−06 | 1.07526E−05 | 3.387270544 | 9.23451729 |
| | secretion | D0897_02275 | | | | | | | |
| | protein | | | | | | | | |
| R4Y4I7_KLEPR | HmsF protein | hmsFKPR_0497 | Klebsiella pneumoniae | 671 | UniRef90_R4Y4I7 | 1.83297E−08 | 8.82339E−06 | 8.911008581 | 458.872816 |
| | | | subsp. rhin [symbol] | | | | | | |
| B5Y1W1_KLEP3 | Leucineopreon | leuL KPK_4661 | Klebsiella pneumoniae | 28 | UniRef90_B5Y1W1 | 6.17438E−07 | 8.75217E−06 | 3.825275514 | 15.6165621 |
| | leader | | (strain 342 [symbol] | | | | | | |
| | peptide | | | | | | | | |
| W0BTZ6_ENTCL | LysR family | M942_15825 | Enterobacter cloacae P101 | 305 | UniRef90_W0BTZ6 | 3.73738E−07 | 9.12235E−06 | 4.609307144 | 24.6607934 |
| | transcriptional | | | | | | | | |
| | regulator | | | | | | | | |
| E1KWK7_FINMA | Metallo-beta- | HMPREF9289_0746 | Finegoldia magna BVS033A4 | 240 | UniRef90_E1KWK7 | 6.20507E−10 | 2.60972E−07 | 8.716228635 | 1096.3648 |
| | lactamse | | | | | | | | |
| | domain protein | | | | | | | | |
| W9BI79_KLEPN | MFS transporter | BN49_3651 | Klebsiella pneumoniae | 395 | UniRef90_W9BI79 | 1.01277E−06 | 1.05023E−05 | 3.374327932 | 10.006729 |
| | | D0897_02300 | | | | | | | |
| A7KFZ3_KLEPN | Nickel/cobalt | [symbol] rcnA_2 | Klebsiella pneumoniae | 371 | UniRef90_A7KFZ3 | 5.73881E−07 | 1.29273E−05 | 4.493521068 | 16.9842372 |
| | efflux | B4U30_02080 | | | | | | | |
| | system | SAME [symbol] | | | | | | | |
| C3R370_9BACE | Nucleic acid- | BSCG_05583 | Bacteroides sp. 2_2_4 | 127 | UniRef90_C3R370 | 1.55708E−09 | 1.60514E−05 | 13.33156903 | 7908.44248 |
| | binding | | | | | | | | |
| | domain protein | | | | | | | | |
| B5XVF2_KLEP3 | PAP2 family | KPK_1137 | Klebsiella pneumoniae | 198 | UniRef90_B5XVF2 | 1.78181E−07 | 1.65687E−05 | 6.538970419 | 199.105431 |
| | protein | | (strain 342 [symbol] | | | | | | |
| F8HFC6_STRE5 | Permease family | Ssal_00258 | Streptococcus salivarius | 668 | UniRef90_F8HFC6 | 3.78436E−10 | 4.60234E−07 | 10.24810464 | inf |
| | protein | | (strain 57 [symbol] | | | | | | |
| D7IXQ4_9BACE | Ribosephosphate | HMPREF0104_04250 | Bacteroides sp. 3_1_19 | 188 | UniRef90_D7IXQ4 | 4.00423E−10 | 1.61529E−05 | 15.29991329 | 28834.2232 |
| | pyrophosphokinase | | | | | | | | |
| G8LGA3_ENTCL | Serine/threonine- | pphA EcWSU1_02763 | Enterobacter cloacae EcWSU1 | 233 | UniRef90_G8LGA3 | 6.50725E−08 | 4.61242E−06 | 6.147332599 | 349.404297 |
| | protein | | | | | | | | |
| | phosphate 1 | | | | | | | | |
| C3R379_9BACE | Single-stranded | BSCG_05592 | Bacteroides sp. 2_2_4 | 132 | UniRef90_C3R379 | 1.33793E−08 | 7.51935E−06 | 9.134464192 | 293.938698 |
| | DNA-binding | | | | | | | | |
| | protein | | | | | | | | |
| E1KWK6_FINMA | Single-stranded | HMPREF9289_0745 | Finegoldia magna BVS033A4 | 144 | UniRef90_E1KWK6 | 1.83383E−09 | 2.9436E−07 | 7.326581684 | 132.404888 |
| | DNA-binding | | | | | | | | |
| | protein (SSB) | | | | | | | | |
| D7IXQ0_9BACE | Toxin-antitoxin | HMPREF0104_04246 | Bacteroides sp. 3_1_19 | 192 | UniRef90_D7IXQ0 | 4.85835E−10 | 3.86025E−06 | 12.95593995 | 6273.04844 |
| | system, | | | | | | | | |
| | toxin component, | | | | | | | | |
| | Hip [symbol] | | | | | | | | |
| F8LLC4_STREH | Transcriptional | degU | Streptococcus salivarius | 194 | UniRef90_F8LLC4 | 8.71036E−10 | 5.62499E−07 | 9.334902218 | inf |
| | regulatory | SALIVB_1891 | (strain [symbol] | | | | | | |
| | protein degU | | | | | | | | |
| | (Prote [symbol] | | | | | | | | |
| Y4780_KLEP3 | UPF0391 | KPK_4780 | Klebsiella pneumoniae | 53 | UniRef90_Y4780 | 0.000008 | 0.000149 | 4.180712 | 18.1350957 |
| | membraneprotein | | (strain 342 [symbol] | | | | | | |
| | KPK_4780 | | | | | | | | |

[symbol]

indicates data missing or illegible when filed

TABLE 6
The most important genes that distinguish NEC from control preterm infants that are mobile elements.

| | | | | | | Healthy | NEC | Log2 | Fold |
|---|---|---|---|---|---|---|---|---|---|
| ID | Protein names | Gene names | Organism | Length | ID_proc | [symbol] | mean | FC | Change |
| I4S9D1_ECOLX | Antirepressor | EC54115_22298 | Escherichia coli 541-15 | 324 | UniRef90_I4S9D1 | 8.37421E−09 | 1.91583E−06 | 7.83780088 | 192.029619 |
| | protein | | | | | | | | |
| F4TMD8_ECOLX | Transposase | ECJG_05326 | Escherichia coli M718 | 47 | UniRef90_F4TMD8 | 6.87104E−11 | 2.33375E−08 | 8.407906678 | inf |
| | for insertion | | | | | | | | |
| | sequence element | | | | | | | | |
| H6LBS8_ACEWD | Type I restriction- | hsdM2 Awo_c08800 | Acetobacterium woodii | 506 | UniRef90_H6LBS8 | 0 | 2.16125E−07 | #DIV/0 [symbol] | inf |
| | modification | | (strain AT [symbol] | | | | | | |
| | system methylt [symbol] | | | | | | | | |
| S0NHM8_9ENTE | Type I restriction- | OMQ_01160 | Enterococcus saccharolyticus | 507 | UniRef90_S0NHM8 | 0 | 2.6782E−07 | #DIV/0 [symbol] | inf |
| | modification | | subs [symbol] | | | | | | |
| | system, Msubu | | | | | | | | |
| Q64WL9_BACFR | Conserved protein | BF1360 | Bacteroides fragilis | 111 | UniRef90_Q64WL9 | 3.76321E−10 | 6.92859E−06 | 14.16830849 | inf |
| | found in conjugate | | (strain YCH46) | | | | | | |
| | transpos [symbol] | | | | | | | | |
| Q64WM1_BACFR | Conserved protein | BF1357 | Bacteroides fragilis | 208 | UniRef90_Q64WM1 | 7.53066E−10 | 5.17451E−06 | 12.74635959 | 1466.5704 |
| | found in conjugate | | (strain YCH46) | | | | | | |
| | transpo [symbol] | | | | | | | | |
| Q64WM9_BACFR | Conserved protein | BF1348 | Bacteroides fragilis | 152 | UniRef90_Q64WM9 | 2.95772E−10 | 5.79943E−06 | 14.25914007 | 32533.9868 |
| | found in conjugate | | (strain YCH46) | | | | | | |
| | transpos [symbol] | | | | | | | | |
| D4VS09_9BACE | Conjugate transposon | BV890_15910 | Bacteroides xylanisolvens | 251 | UniRef90_D4VS09 | 2.1E−09 | 5.70519E−06 | 11.40767069 | 1865.31989 |
| | protein TraA | | SDCC1 [symbol] | | | | | | |
| W1YJ73_9ZZZZ | CRISPR-associated | Q604_UNBC03640G001 | human gut metagenome | 96 | UniRef90_W1YJ73 | 1.9241E−09 | 1.06715E−06 | 9.11539299 | 974.644143 |
| | protein, Csm1 | | | | | | | | |
| | family (Fragm [symbol] | | | | | | | | |
| B7T0C8_9CAUD | Gp38 | | Stapylococcus virus IPLA88 | 61 | UniRef90_B7T0C8 | 1.42918E−09 | 2.22336E−06 | 10.60334515 | 1261.71358 |
| D6D3M1_98ACE | Homologues of | BXY_41020 | Bacteroides xylanisolvens | 333 | UniRef90_D6D3M1 | 9.16702E−10 | 7.03671E−06 | 12.90616058 | 10576.1775 |
| | Tra [symbol] from | | XB1A | | | | | | |
| | Bacteroides | | | | | | | | |
| | conjugat [symbol] | | | | | | | | |
| G8I0W8_STAAU | Integrase | int | Staphyloccus aureus | 372 | UniRef90_G8IDW8 | 3.70507E−09 | 6.14426E−06 | 10.69552152 | 7045.00533 |
| B5XPQ3_KLEP3 | Integrase | KDK_1799 | Klebsiella pneumoniae | 416 | UniRef90_B5XPQ3 | 5.07273E−09 | 5.3421E−07 | 6.718501267 | 122.592651 |
| | | | (strain 342 [symbol] | | | | | | |
| C1UI5_ENTCL | Integrase | AM401_24355 | Enterbacter cloacae | 174 | UniRef90_C1IUI5 | 4.97175E−07 | 9.30317E−06 | 4.225898404 | 14.9196251 |
| | | B9Q36_1807 [symbol] | | | | | | | |
| G2SBG8_ENTAL | Integrase family | Entas_2732 | Enterobacter asburiae | 430 | UniRef90_G2SBG8 | 1.45814E−07 | 1.26977E−05 | 6.44429197 | 61.5610093 |
| | protein | | (strain LF7a [symbol] | | | | | | |
| Q8SDU9_BPPHA | Large terminase | | Staphylococcus phage | 447 | UniRef90_Q8SDU9 | 7.25391E−09 | 6.30427E−06 | 9.763354107 | 730.058478 |
| | | | phi11 (Bact [symbol] | | | | | | |
| Q4ZDW4_9CAUD | ORF044 | | Staphylococcus virus 187 | 120 | UniRef90_Q4ZDW4 | 5.36998E−10 | 6.83169E−06 | 13.63503915 | 10283.9611 |
| Q8SDM3_BPPHD | Phi ETA orf | | Staphylococcus phage | 183 | UniRef90_Q8SDM3 | 1.29175E−09 | 6.47892E−06 | 12.29220553 | 4186.01318 |
| | 18-like protein | | phi13 (Bact [symbol] | | | | | | |
| Q8SDT6_BPPHA | Phi ETA orf | | Staphylococcus phage | 315 | UniRef90_Q8SDT6 | 4.35455E−09 | 5.56863E−06 | 10.32058565 | 1054.54001 |
| | 54-like protein | | phi11 (Bact [symbol] | | | | | | |
| Q8SDL2_BPPHD | Phi PVL orf | | Staphylococcus phage | 150 | UniRef90_Q8SDL2 | 1.69353E−08 | 6.58427E−06 | 8.602846183 | 1829.91391 |
| | 62-like protein | | phi13 (Bact [symbol] | | | | | | |
| Q8SDK9_BPPHD | Portal protein | | Staphylococcus phage | 441 | UniRef90_Q8SDK9 | 1.8566E−08 | 6.20568E−06 | 8.384785851 | 817.415235 |
| | | | phi13 (Bact [symbol] | | | | | | |
| G8LEP_ENTCL | Prophage Tail Protein | EcWSU1_03863 | Enterobacter cloacae EcWSU1 | 39 | UniRef90_G8LEP9 | 6.6919E−08 | 2.02528E−06 | 4.919559955 | 29.227718 |
| Q4QKD1_HAEI8 | Putative recombination | [symbol] ninGNTH1728_1 | Haemophilus influenzae | 129 | UniRef90_Q4QKD1 | 6.72304E−09 | 7.42307E−07 | 6.786757133 | 107.079228 |
| | protein NinG | | (strain 86 [symbol] | | | | | | |
| | homolo [symbol] | | | | | | | | |
| E1KW06_FINMA | Recombinase, phage | HMPREF9_0747 | Finegoldia magna BVS033A4 | 285 | UniRef90_E1KW06 | 1.31945E−09 | 3.49236E−07 | 8.048122947 | 285.388695 |
| | RecT family | | | | | | | | |
| C3R3C3_9BACE | Relaxase/mobilization | B5CG_05636 | Bacteroides sp. 2_2_4 | 466 | UniRef90_C3R3C3 | 1.90211E−08 | 1.63462E−05 | 9.74713694 | 454.496046 |
| | nuclease domain | | | | | | | | |
| | pretei [symbol] | | | | | | | | |
| Q8SDV0_BPPHA | Small terminase | | Staphylococcus phage | 146 | UniRef90_Q8SDV0 | 4.37844E−09 | 6.76293E−06 | 10.59301763 | 1245.59813 |
| | | | phi11 (Bact [symbol] | | | | | | |
| Q9MBQ2_8PPHD | Terminase-large | | Staphylcoccus phage | 564 | UniRef90_Q9MBQ2 | 1.80704E−08 | 6.09913E−06 | 8.398829897 | 639.622995 |
| | subunit | | phi13 (Bact [symbol] | | | | | | |
| G8LF67_ENTCL | Ych0 | ych0EcWSU1_02617 | Enterobacter cloacae EcWSU1 | 481 | UniRef90_G8LF67 | 3.78071E−07 | 8.46681E−06 | 4.485087358 | 22.3287507 |
| Q77FU2_BPPHD | CI-like repressor | | Staphylcoccus phage | 256 | UniRef90_Q77FU2 | 6.2093E−09 | 3.78988E−06 | 9.253504671 | 1135.65095 |
| | | | phi13 (Bact [symbol] | | | | | | |

[symbol]

indicates data missing or illegible when filed

Legend for Table 5 and 6. The tables shows the most important microbial genes that were identified by the model to discriminate between NEC and controls. ID=UniProt gene ID; Protein names=UniProt protein name; Gene names=UniProt gene name; Organism=The taxonomic affiliation of the gene; Length=The protein length in aa; ID_proc=Uniref_90 ID; Healthy preterm mean=Mean value of the gene in CPM (copy per million); NEC mean=Mean value of the gene in CPM (copy per million); Log2 FC=The Log2 fold change difference of CPM values between NEC and controls. Fold change is the mean value NEC/mean value healthy preterm control. If these genes reported in the table are removed from the input, this will cause the collapse of the predictive model, namely the model would not be able to discriminate between NEC and controls with any meaningful accuracy that is more than random guessing. Therefore, the listed genes are the most influential genes that appear to be always higher in the NEC samples compared to controls. The genes are ranked based on their importance in the model, in terms of predictiveness of NEC (Table 7).

To determine the minimum number of samples required for training an informative model, a random forest classifier was trained on a random subset of features. The mean accuracy was obtained for each samples size. With even class distribution, a minimum number of 30 samples would begin to yield minimum discriminatory power. Optimally, it was determined that approximately 10,000 features would best eliminate overfitting, however approximately 1,000 features would yield sufficient explanatory power for treatment purposes.

TABLE 7
Top 72 Features from Recursive Feature Elimination Ranking. These
represent the minimum number of features that reliably obtained the
highest accuracy seen on the training and testing datasets.

| Rank | Feature |
|---|---|
| 1 | UniRef90_G2SBG8 |
| 2 | UniRef90_B5XVF2 |
| 3 | UniRef90_Q8SDM3 |
| 4 | UniRef90_D7IXQ4 |
| 5 | UniRef90_X8H364 |
| 6 | UniRef90_B5XPQ3 |
| 7 | UniRef90_G2S602 |
| 8 | UniRef90_G8I0W8 |
| 9 | UniRef90_W1GNF8 |
| 10 | UniRef90_Q64WL9 |
| 11 | UniRef90_W1E8N6 |
| 12 | UniRef90_Q8SDU9 |
| 13 | UniRef90_S0NHM8 |
| 14 | UniRef90_F8LLC4 |
| 15 | UniRef90_G8LGA3 |
| 16 | UniRef90_A0A017N0P3 |
| 17 | UniRef90_W5VJZ3 |
| 18 | UniRef90_A7KFZ3 |
| 19 | UniRef90_B5Y280 |
| 20 | UniRef90_H6LBS8 |
| 21 | UniRef90_D6D3M7 |
| 22 | UniRef90_Q4ZDW4 |
| 23 | UniRef90_F8HFC6 |
| 24 | UniRef90_C3R370 |
| 25 | UniRef90_W0BTZ6 |
| 26 | UniRef90_Q8SDV0 |
| 27 | UniRef90_I4S9D1 |
| 28 | UniRef90_Q77FU2 |
| 29 | UniRef90_D4VS09 |
| 30 | UniRef90_W1HIJ7 |
| 31 | UniRef90_Q4QKD1 |
| 32 | UniRef90_W1DIL6 |
| 33 | UniRef90_W9BI79 |
| 34 | UniRef90_Q64WM9 |
| 35 | UniRef90_G8LMZ9 |
| 36 | UniRef90_E1L414 |
| 37 | UniRef90_W1DZS6 |
| 38 | UniRef90_E1KW06 |
| 39 | UniRef90_A7KFV2 |
| 40 | UniRef90_G8LF67 |
| 41 | UniRef90_B7T0C8 |
| 42 | UniRef90_A0A015XHM2 |
| 43 | UniRef90_Q8SDT6 |
| 44 | UniRef90_D6D3M1 |
| 45 | UniRef90_W1H3V7 |
| 46 | UniRef90_W9BPS5 |
| 47 | UniRef90_A7KFW2 |
| 48 | UniRef90_A7MFQ2 |
| 49 | UniRef90_P01553 |
| 50 | UniRef90_C3R3C3 |
| 51 | UniRef90_Q9MBQ2 |
| 52 | UniRef90_E1KWK6 |
| 53 | UniRef90_C3RFZ0 |
| 54 | UniRef90_P15236 |
| 55 | UniRef90_W1G6G6 |
| 56 | UniRef90_G8LJG5 |
| 57 | UniRef90_R4Y4I7 |
| 58 | UniRef90_W9BAX7 |
| 59 | UniRef90_C1IUI5 |
| 60 | UniRef90_G8LEP9 |
| 61 | UniRef90_A7MQQ8 |
| 62 | UniRef90_Q64WM1 |
| 63 | UniRef90_F4TMD8 |
| 64 | UniRef90_Q8SDL2 |
| 65 | UniRef90_W1YJ73 |
| 66 | UniRef90_W1EGX2 |
| 67 | UniRef90_Q8SDK9 |
| 68 | UniRef90_b5Y1W1 |
| 69 | UniRef90_C3R379 |
| 70 | UniRef90_A7KFV8 |
| 71 | UniRef90_D7IXQ0 |
| 72 | UniRef90_E1KWK7 |

Each model was used to obtain the percent risk of each sample classifying as NEC positive. Treatment courses could then be taken to minimize risk of samples developing NEC based on a high risk of between 20 and 50%.

In some embodiments of this invention the risk for NEC is determined by the detection and/or quantification of the biomarkers listed on Table 7 or any combinations thereof. In preferred embodiments of this invention NEC risk is determined based on the detection and/or quantification of any combination of the UniRef90_G2SBG8, UniRef90_B5XVF2, UniRef90_Q8SDM3, UniRef90_D71XQ4, UniRef90_X8H364, UniRef90_B5XPQ3, UniRef90_G2S602, UniRef90_G810W8, UniRef90_W1GNF8, UniRef90_Q64WL9 biomarkers, or homologues thereof. In more preferred embodiments of this invention determination of the risk of NEC can be made by the detection and/or quantification of the following biomarkers or, homologues thereof, and/or the presence of an organism associated with the detection of the relevant biomarker as follows: UniRef90_G2SBG8 an integrase family protein associated with Enterobacter asburiae; UniRef90_B5XVF2 a PAP2 family protein associated with Klebsiella pneumoniae; UniRef90_Q8SDM3 a phi ETA irf 18-like protein associated with Staphylococcus phage phi13; UniRef90_D71XQ4 a ribose phosphate pyrophosphokinase associated with Bacteroides sp.; UniRef90_X8H364 an arylsulfatase associated with Veillonella sp.

In some embodiments of this invention the risk of NEC may be determined by the presence/absence and/or the quantification of any combination of microbial organisms enumerated on Table 5 and Table 6. In preferred embodiments of this invention determination of the risk for NEC can be made by the detection and/or quantification of Klebsiella spp., Veillonella spp., Bacteroides spp., Enterobacter spp., Bacteriophage phi-13, Bacteriophage phi-11, or any combination thereof. In preferred embodiments of this invention the risk of NEC may be determined by the presence/absence and/or quantification of Klebiella pneumonia, Enterobacter asburiae, Bacteroides fragilis, Viellonella sp. ICM51a, Bacteriophage-13, and/or Bacteriophage phi-11 or any combination thereof.

Biomarkers identified by this process can be used to diagnose and monitor infants in the NICU to highlight dysbiosis, indicate dysfunction, and predict risk factors to stratify infants and treat the underlying dysbiosis and/or dysfunction through therapies designed to treat the observed dysbiosis. In some cases the therapy may include the addition of Bifidobacterium and more specifically B. infantis to reverse dysbiosis in these preterm infants. Therapeutic steps for this invention are described in WO 2016/065324, WO 2016/149149, WO 2017/156550, and WO 2018/006080, incorporated herein by reference.

This information may also be used to target antimicrobial therapies that can target microbial pathway without interfering with host metabolic pathways, or those of beneficial bacteria.

### Clinical Uses

The invention can be used to evaluate any microbiome associated with the body including but not limited to the vaginal, gut, skin, buccal, milk, or other surfaces that have a specific microbiome that might be implicated in NEC. Surfaces in the environment may also be evaluated for their contribution of virus, bacteria, mold and/or yeast. In some embodiments, one or more of the microbiome in the preterm or term infant or surrounding the preterm or term infant is used as part of the AI model. In other embodiments, host data including anthropometry, blood work, fecal cytokines, fecal calprotectin, T cell profiles may also be used in an AI model to evaluate success of altering risk profile for preterm infants born into specific hospital systems to assess risk of NEC.

To assess risk to the preterm infant, a particular group may also be monitored as a group residing in a particular part of the hospital or health care system such as, but not limited to hospitalized patients in the neonatal intensive care unit, the pediatric intensive care unit, the intensive care unit for non-pediatric patients i.e., adults, the emergency room, the cardiology unit, psychiatric unit, or the neurology unit in which bacteria containing the elements of. It may also be applied to specific outpatient facilities with particular risks including infections and more particularly antibiotic resistant infections are known, but best treatment strategy is unknown.

Machine learning as described herein may be used to understand the dispersion of antibiotic resistance genes across a health system and/or geographic region, to understand risk and provide data driven strategies to improve antibiotic stewardship and/or to understand the emergence of new resistance and/or to understand the full resistome to better prescribe antibiotics to reduce treatment failure in NEC.

A dashboard or a system of assessing risk that provides a tool for a clinician to monitor the health of a preterm infant to alter and/or implement a treatment regime who is at particular risk of a condition or disease based on the environment they find themselves in, their genetic predisposition to particular conditions or have pre-clinical presentation of risk that is a precursor to overt symptoms (i.e intestinal integrity).

A subset of proteins, enzymes, peptides, metabolites can be monitored to to inform clinician of risk selected from Table 5 and/or 6.

The genes identified in Tables 5 and 6 may be monitored with a PCR method that amplifies one or more genes from Table 5 or 6 using specific validated primers to look for fold changes. Inflammatory markers such as calprotectin or fecal cytokines may be monitored. ATP or lactate dehydrogenase levels may also be monitored.

The embodiments, of this test may be used to improve known treatment, and ensure that treatment is effective in reducing the presence of the organisms and genes identified in Table 5 and 6. The introduction of B. infantis in a diet that contains human milk oligosaccharides or their functional equivalents is one such treatment for the prevention or reduction in risk for NEC. Premature infant treatment is complicated by routine antibiotic use and other medicines that may render addition of probiotics and prebiotics to improve microbiome function less effective. In an embodiment, a B. infantis alone or in combination with other probiotic bacteria are used as part of the standard of care. In a preferred embodiment, Bifidobacterium longum subsp. infantis may comprise a functional H5 gene cluster (genes required for successful colonization of the infant gut), including Bifidobacterium longum subsp. infantis EVC001 deposited under ATCC Accession No. PTA-125180 (“Deposited Bifidobacterium”).

### Example 1. Hospital Wide Applications for Repeated Use of the Algorithm to Assess Risk

Hospitals have the opportunity to assess risk based on banked fecal samples in different hospital units. A cohort may be established that analyzes the metagenomes of all hospitalized individuals within that cohort, separated into those that developed disease and those that did not, or those that responded to treatment and the non-responders to a given treatment. The analysis provides an output of major taxa, superpathways, metabolites enzyme activities, or proteins associated with disease risk. In that particular unit for that particular condition, a treatment plan or protocol can be implemented aimed at eliminating a key risk factor. The success of the treatment, processes or protocol may be assessed by collecting samples from the cohort post-change in practice. The post-change cohort validates the success of the reduction in risk associated with specific treatments, protocols or processes.

The above may be applied to environmental monitoring of hospital environments for key taxa associated with NEC. If klebsiella was identified as a key risk in a specific hospital environment, a new cleaning protocol would be implemented that was known to reduce klebsiella on hospital surfaces in order to reduce transmission to the infant. Following a set time frame, new fecal samples are taken to assess the success of an intervention. Machine learning requires minimum of 30 independent samples to assess the success of any given treatment.

### Example 2. Evaluation of Intestinal Integrity with Altered Microbial Functions

Intestinal integrity is considered a risk factor for many disease conditions including NEC and late onset-sepsis. Leaky gut results when there is insufficient intestinal integrity.

B. infantis EVC001 dominant microbiome produces metabolites improve enterocyte proliferation in vitro.

Short chain fatty acids (SCFA) are an important energy source for host cells to maintain homeostasis. Indeed, SCFAs account for 50-70% of the energy used by intestinal epithelial cells (IECs) and provide nearly 10% of our daily caloric requirements. Given previous findings showing infants colonized with B. infantis EVC001 have significantly increased fecal SCFAs concentrations compared to infants not colonized with B. infantis, we investigated the effect of fecal water (FW) from two distinct populations on enterocyte proliferation and morphology in vitro.

Fecal Waters (FW) were derived from fecal samples from infants colonized with B. infantis EVC001 (EVC001) and infants not colonized with B. infantis (controls). FW were added to adult and premature enterocyte cell lines to assess growth, proliferation and cytotoxicity. Microscopic images were taken to observe morphological differences.

Intestinal epithelial cells (Caco-2 and HIEC-6 cells) exposed to EVC001 FW showed significantly increased proliferation as shown by cell count and real-time ATP expression compared to medium alone and control FW (P<0.0001). Conversely, significantly decreased lactate dehydrogenase, an indication of decreased membrane integrity, was detected in enterocytes exposed to EVC001 FW compared to controls FW (P<0.01). Furthermore, control FW altered the morphology of enterocytes compared to cells exposed to EVC001 FW or medium alone.

EVC001 FW significantly increased enterocyte proliferation compared to control FW and medium alone, while control FW negative affected cell growth, membrane integrity and cell morphology; thus, suggesting SCFA produced by B. infantis EVC001 promote enterocyte growth and improve intestinal integrity in infants.

This in vitro model is applicable to assess the effect of any of the metabolites identified herein, but specifically the evaluation of fecal waters with microbiomes expected to deplete ARG on intestinal integrity. The addition of supplemental arginine can be investigated. This model may be used to evaluate fecal waters from healthy preterm infants, those supplemented with B. infantis and those with NEC. This model may also be used to evaluate the effect of specific inhibitors of microbial arginine pathways to limit the growth of those organisms. This method can be used to help develop new targeted antimicrobials against the bacteria specifically implicated in NEC.

1. A method of determining risk of necrotizing enterocolitis (NEC) in an infant, comprising:
a) obtaining a fecal sample of the infant's relevant microbiome;
b) sequencing genetic material in the sample to obtain sequence data for the relevant microbiome;
c) analyzing sequence data for the relevant microbiome to identify biomarkers in the infant's microbiome; and
d) categorizing the NEC risk of the infant using the biomarkers identified in the microbiome of the infant.

2. The method of claim 1, wherein categorizing according to step (d) is based on an artificial intelligence (AI) model developed by analyzing sequence data from the relevant microbiomes of N infants, said N infants comprising at least M infants diagnosed with NEC, and N−M infants not diagnosed with NEC, said AI model developed by processing the sequence data from the relevant microbiomes of the N infants by Machine Learning algorithms to identify at least X biomarkers which differ significantly between infants diagnosed with NEC and infants not diagnosed with NEC and associating said X biomarkers with infants having or at risk for having NEC.

3. The method of claim 2, wherein N is at least 10-fold higher than X and M is at least 2-fold higher than X.

4. The method of any one of claim 2 or 3, wherein X is at least 5, at least 10, at least 20, at least 30 or at least 40 biomarkers.

5. The method of any one of claims 2-4, wherein Nis between 400 and 10,000 infants, and M is between 200 and 1300 infants.

6. The method of any one of claims 2-4, wherein N is at least 30, at least 50, at least 100, at least 250, at least 500, at least 1000, or at least 10,000 infants.

7. The method of any preceding claim, wherein the biomarkers identified in step (c) are proteins, mobile genetic elements, functional annotations, superpathways, and/or taxonomic identifiers.

8. The method of any preceding claim, wherein the biomarkers identified in step (c) are biomarkers found on Table 5 and/or 6.

9. The method of claim 8, wherein at least 3 biomarkers are selected from the top 20 influencers in the NEC model from Table 7.

10. The method of claim 8, wherein at least 5 biomarkers are selected from the top 20 influencers in the NEC model from Table 7.

11. The method of any preceding claim, wherein the infant is a term infant or a preterm infant.

12. The method of any preceding claim, wherein the relevant microbiome is an intestinal microbiome, fecal microbiome, a milk microbiome, a skin microbiome, an environmental microbiome, or a combination thereof.

13. The method of any preceding claim, further wherein the infant's risk for NEC is categorized as high based on the presence of any biomarker enumerated in Table 7. The method of any preceding claim, further wherein the infant's risk of NEC is categorized as high if intestinal arginine levels are at least 1 fold lower compared to known intestinal arginine levels of preterm infants who do not get NEC, Fecal ATP levels, if fecal calprotectin is higher, if lactate dehydrogenase is increased compared to preterm infants who do not get NEC, and/or the

14. The method of ant of the preceding claims Wherein the risk of NEC is at least 5 fold higher for any of the gene abundance of biomarkers identified in Table 5 and 6 compared to the reference control infants.

15. The method of any preceding claim, further wherein the infant's risk for NEC is categorized as high based on the presence of any biomarker enumerated in Table 7.

16. The method of any preceding claim wherein the infant's risk for NEC is categorized as high based on the presence, individually or in any combination, of any of the following biomarkers or homologues thereof: UniRef90_G2SBG8, UniRef90_B5XVF2, UniRef90_Q8SDM3, UniRef90_D71XQ4, UniRef90_X8H364, UniRef90_B5XPQ3, UniRef90_G2S602, UniRef90_G810W8, UniRef90_W1GNF8, UniRef90_Q64WL9.

17. The method of claim 15 wherein the infant's risk for NEC is categorized as high based on the presence of at least 3 of the biomarkers enumerated therein, or homologues thereof.

18. The method of claim 15 wherein the infant's risk for NEC is categorized as high based on the presence of at least 5 of the biomarkers enumerated therein, or homologues thereof.

19. The method of any preceding claim wherein the infant's risk for NEC is categorized as high based on the presence, individually or in any combination, of any of the following bacterial taxa: Klebsiella pneumonia, Enterobacter asburiae, Bacteroides fragilis, Viellonella sp., Bacteriophage phi-13, and/or Bacteriophage phi-11.

20. The method of any preceding claim wherein an infant having risk of NEC categorized as high is treated by administering B. infantis and/or mammalian milk oligosaccharides (MMO).
